(19) United States
(12) Patent Application Publication
(10) Pub. No.: US 2007/0191516 A1
(43) Pub. Date: Aug. 16, 2007

(54) Process for the synthesis of amine ethers

(76) Inventors: Markus Frey, Rheinfelden (CH); Valerie Rast, Basel (CH); Adalbert Braig, Binzen (DE); Andreas Kramer, Meyriez (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/591,778

(22) PCT Filed: Mar. 7, 2005
(86) PCT No.: PCT/EP05/50995

§ 371 Date: Sep. 6, 2006

(30) Foreign Application Priority Data
Mar. 15, 2004 (EP) 04101047.1

(51) Int. Cl. C08K 5/34; C07D 403/14; C07D 401/14

(52) U.S. Cl. 524/99; 544/209; 546/16; 546/186; 546/216; 546/14; 544/129; 524/102

(57) ABSTRACT

A process for the preparation of a sterically hindered amine ether which comprises reacting a corresponding sterically hindered aminoxide with a C₅-C₁₈alk-1-ene in the presence of an organic hydroperoxide and optionally hydrogenating the resulting product as well as the product mixtures obtained therewith and their use as stabilizers and flame retardants.

The instant invention pertains to a process for preparing sterically hindered amine ethers, e.g. N-hydrocarbyloxy substituted sterically hindered amine compounds and mixtures thereof, by the reaction of the corresponding N-oxyl intermediate with a 1-alkene in the presence of an organic hydroperoxide, optionally together with a further catalyst, and some novel compound mixtures obtainable by this process. The instant invention also pertains to a process of hydrogenating unsaturated amine ethers, and some novel compound mixtures obtainable by this process. The compounds made by the processes are particularly effective in the stabilization of polymer compositions against harmful effects of light, oxygen and/or heat and as flame-retardants for polymers.

WO 01/92228 describes a process for the preparation of amine ethers, e.g. N-hydrocarbyloxy substituted hindered amine compounds, by the reaction of the corresponding N-oxyl intermediate with a hydrocarbon in the presence of an organic hydroperoxide and a copper catalyst.

WO 03/045919 describes a process for the preparation of amine ethers, e.g. N-hydrocarbyloxy substituted hindered amine compounds, by the reaction of the corresponding N-oxyl intermediate with a hydrocarbon in the presence of an organic hydroperoxide and an iodide catalyst.

It has now been found that N-alk-2-enyloxy substituted sterically hindered amine ethers and mixtures thereof can most suitably be prepared from N-oxyl intermediate and a 1-alkene in the presence of an organic hydroperoxide and optionally a further catalyst. The process of the invention does not require high reaction temperature. The corresponding N-alkoxy substituted sterically hindered amines can be prepared from said N-alk-2-enyloxy substituted sterically hindered amines by hydrogenation. With the procedure according to this invention purer products may be obtained. The obtained product mixtures may contain less isomers.

Thus, present invention pertains to a process for the preparation of a sterically hindered amine ether which comprises reacting a corresponding sterically hindered aminoxide with a C₅-C₁₈alk-1-ene in the presence of an organic hydroperoxide.

The present invention further pertains to a process, wherein the sterically hindered amine ether obtained by reacting a corresponding sterically hindered aminoxide with a C₅-C₁₈alk-1-ene in the presence of an organic hydroperoxide is subsequently hydrogenated.

The sterically hindered amine oxide preferably used in the process of the present invention contains at least one group of formula (II) 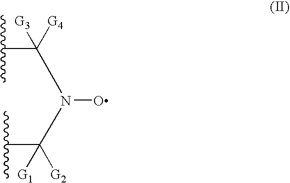
wherein G₁, G₂, G₃ and G₄ are independently alkyl of 1 to 4 carbon atoms or G₁ and G₂ and/or G₃ and G₄ are together tetramethylene or pentamethylene.

[symbol] specifies the border of a chemical group and does not have a chemical meaning by itself.

Advantageously, the sterically hindered amine oxide used in the process of the present invention is a compound of formula (IIa) 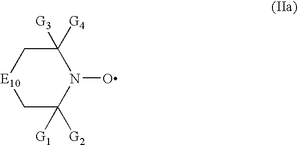
wherein G₁, G₂, G₃ and G₄ are as defined for formula (II);
E₁₀ is a carbon atom which is unsubstituted or substituted by —OH, ═O or by one or two organic residues containing in total 1-500 carbon atoms.

Advantageously, the sterically hindered amine ether obtained according to the present invention contains at least one group of formula (III) 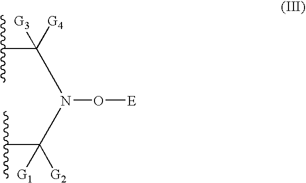
wherein G₁, G₂, G₃ and G₄ are as defined for formula (II) and
E is C₅-C₁₈alkyl or C₅-C₁₈alk-2-enyl.

C₅-C₁₈alkyl as substituent E is preferably a mixture of the radicals 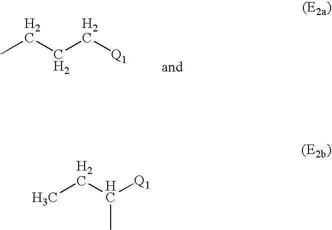
wherein Q₁ is C₂-C₁₅ alkyl;
C₅-C₁₈alk-2-enyl as substituent E is preferably a mixture of the radicals 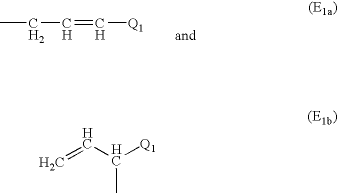
wherein Q₁ is C₂-C₁₅alkyl.

In the context of the description of the present invention, the term C₅-C₁₈alkyl comprises the branched and unbranched isomers of pentyl, hexyl, heptyl, octyl, nonyl, decyl, undecyl, dodecyl, tridecyl, tetradecyl, pentadecyl, hexadecyl, heptadecyl and octadecyl and the term C₂-C₁₆alkyl comprises ethyl and the branched and unbranched isomers of propyl, butyl, pentyl, hexyl, heptyl, octyl, nonyl, decyl, undecyl, dodecyl, tridecyl, tetradecyl and pentadecyl.

The obtained sterically hindered amine ether is preferably a compound of formula (IIIa) 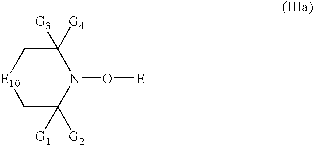
wherein G₁, G₂, G₃ and G₄ are as defined for formula (II);
E is as defined for formula (III);
E₁₀ is as defined for formula (IIa).

Preferably, G₁ and G₃ are methyl and G₂ and G₄ are independently methyl or ethyl.

The obtained sterically hindered amine ether is preferably one of formulae (A) to (O) 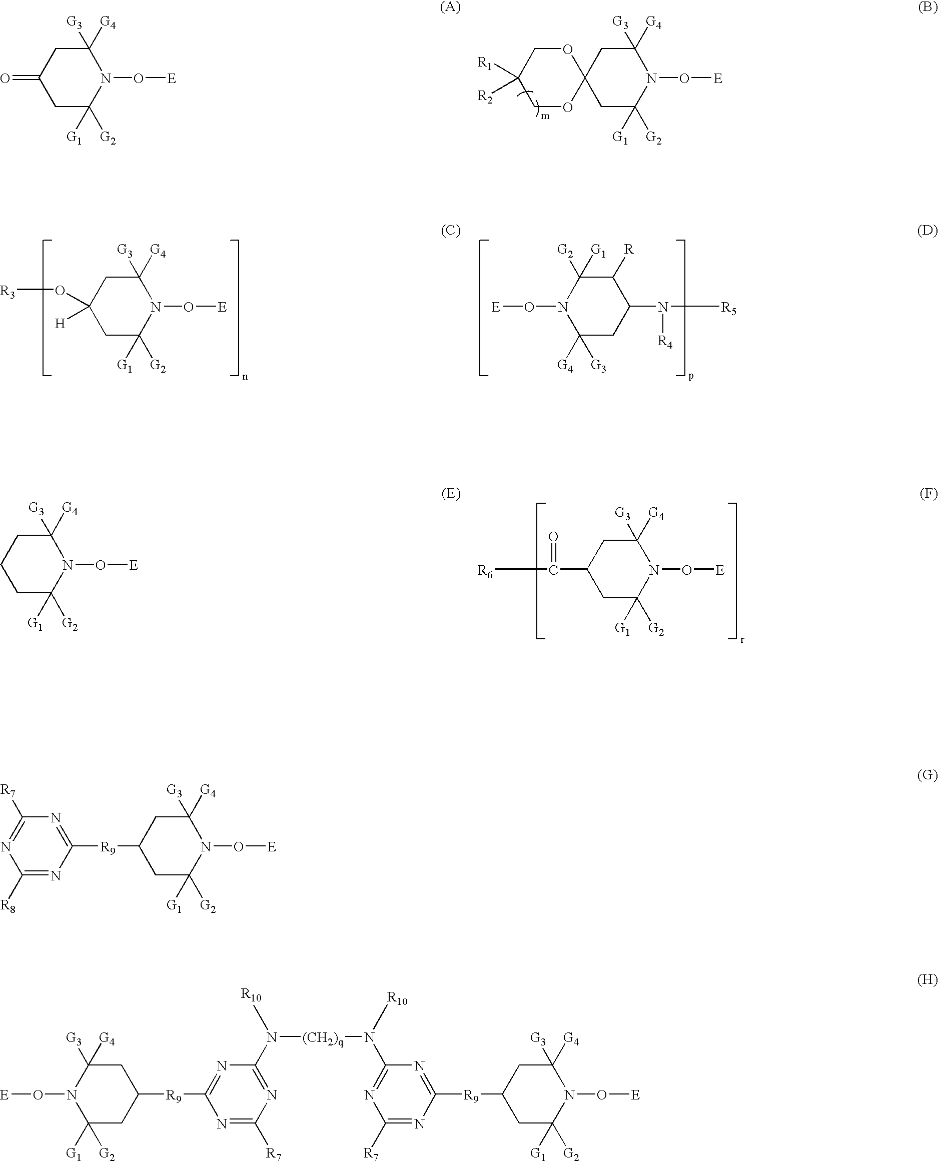 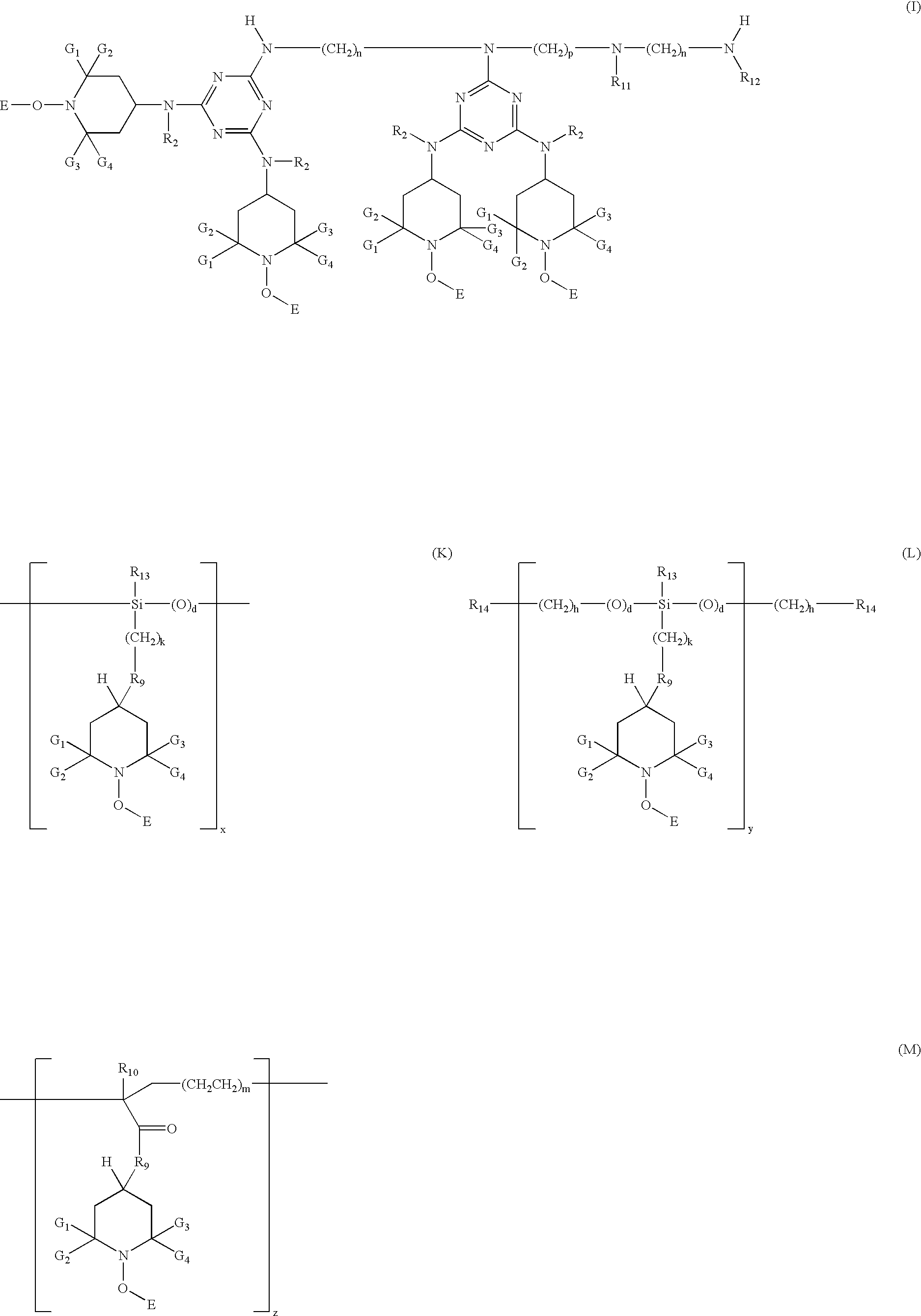 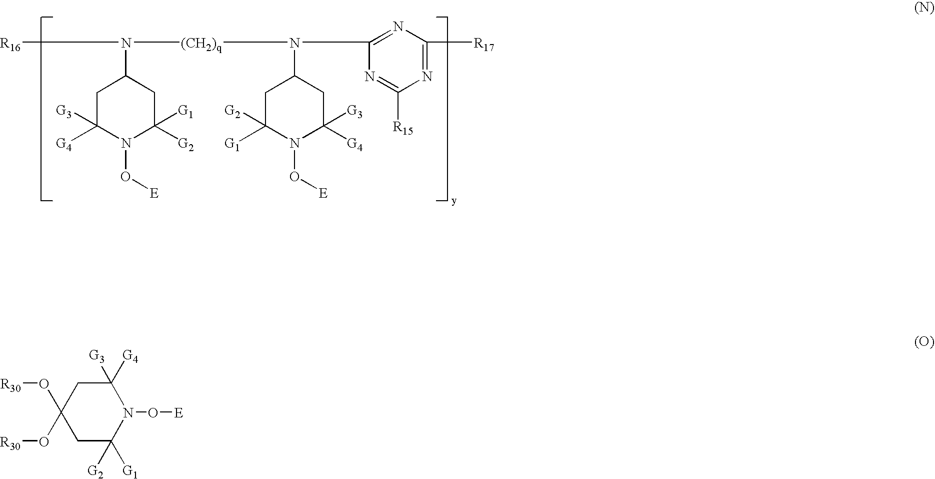
wherein G₁, G₂, G₃ and G₄ are as defined for formula (II);
E is as defined for formula (III);
m is 0 or 1;
R₁ is hydrogen, hydroxyl or hydroxymethyl;
R₂ is hydrogen, alkyl of 1 to 12 carbon atoms or alkenyl of 2 to 12 carbon atoms;
n is 1 to 4;
when n is 1,
R₃ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₃ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₃ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₃ is a tetravalent acyl radical of an aliphatic or unsaturated aliphatic tetracarboxylic acid, especially 1,2,3,4-butanetetracarboxylic acid, 1,2,3,4-but-2-enetetracarboxylic acid, 1,2,3,5-pentanetetracarboxylic acid and 1,2,4,5-pentanetetracarboxylic acid, or R₃ is a tetravalent acyl radical of an aromatic tetracarboxylic acid containing 10 to 18 carbon atoms;
p is 1 to 3,
R₄ is hydrogen, alkyl of 1 to 18 carbon atoms or acyl of 2 to 6 carbon atoms or phenyl;
when p is 1,
R₅ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;
when p is 2,
R₅ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4,
when r is 1,
R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,
when r is 2,
R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₆ is 4-methyl-1,3-phenylenediamino,
when r is 3,
R₆ is a trivalent alkoxy radical of a saturated or unsaturated aliphatic triol containing 3 to 18 carbon atoms,
when r is 4,
R₆ is a tetravalent alkoxy radical of a saturated or unsaturated aliphatic tetraol containing 4 to 18 carbon atoms,
R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms,
R₉ is oxygen, or R₉ is nitrogen substituted by either hydrogen, alkyl of 1 to 12 carbon atoms or T₁,
T₁ is 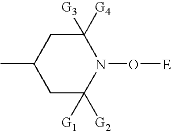
R₁₀ is hydrogen or methyl,
q is 2 to 8,
R₁₁ and R₁₂ are independently hydrogen or the group T₂,
T₂ is 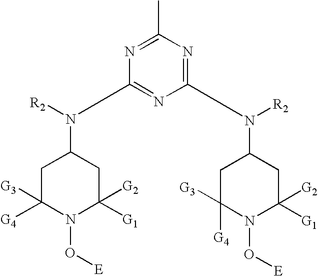
R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₄ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₅ is morpholino, piperidino, 1-piperizinyl, alkylamino of 1 to 8 carbon atoms, especially branched alkylamino of 3 to 8 carbon atoms such as tert-octylamino, —N(alkyl)T₁ with alkyl of 1 to 8 carbon atoms, or —N(alkyl)₂ of 2 to 16 carbon atoms,
R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents may be different;
R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃,
T₃ is 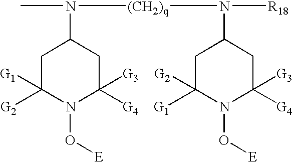
R₁₈ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms;
R₃₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms.

In the definitions of above formulae (A) to (O), the term alkyl comprises, for example, methyl, ethyl and the isomers of propyl, butyl, pentyl, hexyl, heptyl, octyl, nonyl, decyl, undecyl and dodecyl. Examples of alkoxy are methoxy, ethoxy, propoxy, butoxy, octyloxy etc. Examples of alkenyl are vinyl and especially allyl.

Some examples of cycloalkyl are cyclobutyl, cyclopentyl, cyclohexyl, methylcyclopentyl, dimethylcyclopentyl and methylcyclohexyl.

Some examples of an aliphatic carboxylic acid are acetic, propionic, butyric, stearic acid. An example of a cycloaliphatic carboxylic acid is cyclohexanoic acid. An example of an aromatic carboxylic acid is benzoic acid. An example of an aliphatic dicarboxylic acid is malonyl, maleoyl or succinyl, or sebacic acid. An example of a residue of an aromatic dicarboxylic acid is phthaloyl.

An example of a monovalent silyl radical is trimethylsilyl.

Examples of aryl are phenyl and naphthyl. Examples of substituted aryl are methyl-, dimethyl-, trimethyl-, methoxy- or phenyl-substituted phenyl.

Acyl radicals of monocarboxylic acids are, within the definitions, a residue of the formula —CO—R″, wherein R″ may stand inter alia for an alkyl, alkenyl, cycloalkyl or aryl radical as defined. Preferred acyl radicals include acetyl, benzoyl, acryloyl, methacryloyl, propionyl, butyryl, valeroyl, hexanoyl, heptanoyl, octanoyl, nonanoyl, decanoyl, undecanoyl, dodecanoyl, pentadecanoyl, stearoyl. Polyacyl radicals of polyvalent acids are of the formula (—CO)ₙ—R″, wherein n is the valency, e.g. 2, 3, 4, 5 or 6.

Advantageously, the C₅-C₁₈alk-1-ene is an unbranched alkene.

The C₅-C₁₈alk-1-ene is preferably C₆-C₁₂alk-1-ene, in particular C₆-C₈alk-1-ene, for example 1-octene.

The alkylation process of the present invention is preferably carried out in the presence of a further catalyst.

The further catalyst is preferably selected from the group consisting of scandium, titanium, vanadium, chromium, manganese, iron, cobalt, nickel, copper, zinc, gallium, germanium, yttrium, zirconium, niobium, molybdenum, ruthenium, rhodium, palladium, silver, cadmium, indium, tin, antimony, lanthanum, cerium, hafnium, tantalum, tungsten, rhenium, osmium, iridium, platinum, gold, mercury, thallium, lead, bismuth; the compounds thereof; substituted and unsubstituted ammonium iodides and phosphonium iodides.

The further catalyst may also be quaternary ammonium or phosphonium halogenides such as chlorides or bromides. The structure of the ammonium or phosphonium cation is less important; usually, quaternary ammonium or phosphonium cations contain 4 hydrocarbon residues bonded to the central nitrogen or phosphorus atom, which may be, for example, alkyl, phenylalkyl or phenyl groups. Some readily available materials are tetra-C₁-C₁₂alkylated.

The further catalyst may also be any other iodide compound, including organic and inorganic iodide compounds. Examples are alkaline or alkaline earth metal iodides, or onium iodides such as sulfonium iodides, especially quarternary sulfonium iodides. Suitable metal iodides are, inter alia, those of lithium, sodium, potassium, magnesium or calcium.

The further catalyst is more preferably selected from the group consisting of titanium, vanadium, chromium, manganese, iron, cobalt, nickel, copper, zinc, cerium; the halides and oxides thereof; substituted and unsubstituted ammonium iodides and phosphonium iodides.

The further catalyst is most preferably selected from the group consisting of manganese, iron, cobalt, nickel, copper; the halides thereof; substituted and unsubstituted ammonium iodides and phosphonium iodides, for example substituted and unsubstituted quaternary ammonium or phosphonium iodides, especially tetraalkyl ammonium iodides or tetraphenylphosphonium iodide and triphenylalkylphosphonium iodides.

The further catalyst can be bound to an organic or inorganic polymer backbone, rendering a homogenous or heterogeneous catalytic system.

The further catalysts mentioned above may contain anionic ligands commonly known in complex chemistry of transition metals, such hydride ions (H⁻) or anions derived from inorganic or organic acids, examples being halides, e.g. F⁻, Cl⁻, Br⁻ or I⁻, fluoro complexes of the type BF₄⁻, PF₆⁻, SbF₆⁻ or AsF₆⁻, anions of oxygen acids, alcoholates or acetyl ides or anions of cyclopentadiene or oxides.

Anions of oxygen acids are, for example, sulfate, phosphate, perchlorate, perbromate, periodate, antimonate, arsenate, nitrate, carbonate, the anion of a C₁-C₈carboxylic acid, such as formate, acetate, propionate, butyrate, benzoate, phenylacetate, mono-, di- or trichloro- or -fluoroacetate, sulfonates, for example methylsulfonate, ethylsulfonate, propylsulfonate, butylsulfonate, trifluoromethylsulfonate (triflate), unsubstituted or C₁-C₄alkyl-, C₁-C₄alkoxy- or halo-, especially fluoro-, chloro- or bromo-substituted phenylsulfonate or benzylsulfonate, for example tosylate, mesylate, brosylate, p-methoxy- or p-ethoxyphenylsulfonate, pentafluorophenylsulfonate or 2,4,6-triisopropylsulfonate, phosphonates, for example methylphosphonate, ethylphosphonate, propylphosphonate, butylphosphonate, phenylphosphonate, p-methylphenylphosphonate or benzylphosphonate, carboxylates derived from a C₁-C₈carboxylic acid, for example formate, acetate, propionate, butyrate, benzoate, phenylacetate, mono-, di- or trichloro- or -fluoroacetate, and also C₁-C₁₂-alcoholates, such as straight chain or branched C₁-C₁₂-alcoholates, e.g. methanolate or ethanolate. Also oxides are possible.

Anionic ligands and neutral may also be present up to the preferred coordination number of the complex cation of the further catalyst, especially four, five or six. Additional negative charges are counterbalanced by cations, especially monovalent cations such as Na⁺, K⁺, NH₄⁺ or (C₁-C₄ alkyl)₄N⁺.

The further catalysts mentioned above may also contain neutral ligands such as inorganic or organic neutral ligands commonly known in complex chemistry of transition metals. Suitable inorganic ligands are selected from the group consisting of aquo (H₂O), amino, nitrogen, carbon monoxide and nitrosyl. Suitable organic ligands are selected from the group consisting of phosphines, e.g. (C₆H₅)₃P, (i-C₃H₇)₃P, (C₅H₉)₃P or (C₆H₁₁)₃P, di-, tri-, tetra- and hydroxyamines, such as ethylenediamine, ethylenediaminotetraacetate (EDTA), N,N-Dimethyl-N′,N′-bis(2-dimethylaminoethyl)-ethylenediamine (Me₆TREN), catechol, N,N′-dimethyl-1,2-benzenediamine, 2-(methylamino)phenol, 3-(methylamino)-2-butanol or N,N′-bis(1,1-dimethylethyl)-1,2-ethanediamine, N,N,N′,N″,N″-pentamethyldiethyltriamine (PMDETA), C₁-C₈-glycols or glycerides, e.g. ethylene or propylene glycol or derivatives thereof, e.g. di-, tri- or tetraglyme, and monodentate or bidentate heterocyclic e⁻ donor ligands.

The further catalyst can further contain heterocyclic e⁻ donor ligands which are derived, for example, from unsubstituted or substituted heteroarenes from the group consisting of furan, thiophene, pyrrole, pyridine, bis-pyridine, picolylimine, g-pyran, g-thiopyran, phenanthroline, pyrimidine, bis-pyrimidine, pyrazine, indole, coumarone, thionaphthene, carbazole, dibenzofuran, dibenzothiophene, pyrazole, imidazole, benzimidazole, oxazole, thiazole, bis-thiazole, isoxazole, isothiazole, quinoline, bis-quinoline, isoquinoline, bis-isoquinoline, acridine, chromene, phenazine, phenoxazine, phenothiazine, triazine, thianthrene, purine, bis-imidazole and bis-oxazole.

The sterically hindered aminoxides, also referred to as N-oxyl educts for the instant process which include compounds with at least one group of formula (II) or compounds of formula (IIa), are largely known in the art; they may be prepared by oxidation of the corresponding N—H hindered amine with a suitable oxygen donor, e.g. by the reaction of the corresponding N—H hindered amine with hydrogen peroxide and sodium tungstate as described by E. G. Rozantsev et al., in Synthesis, 1971, 192; or with tert-butyl hydroperoxide and molybdenum (VI) as taught in U.S. Pat. No. 4,691,015, or obtained in analogous manner.

The preferred amount of C₅-C₁₈alk-1-ene for the instant process depends, of course, on the relative number of reactive hindered amine nitroxyl moieties in the starting amine oxide. The reaction is typically carried out with a ratio of 1 to 100 moles of C₅-C₁₈alk-1-ene per mole of nitroxyl moiety with the preferred ratio being 1 to 50 moles per mole of nitroxyl moiety, and the most preferred ratio being 1 to 30 moles of C₅-C₁₈alk-1-ene per mole of nitroxyl moiety.

The preferred amount of organic hydroperoxide is 0.5 to 20 moles per mole of nitroxyl moiety, with the more preferred amount being 0.5 to 5 moles of peroxide per mole of nitroxyl moiety and the most preferred amount being 0.5 to 3 moles of peroxide per mole of nitroxyl moiety.

The organic hydroperoxide used in the process of present invention can be of the formula R—OOH, wherein R usually is a hydrocarbon containing 1-18, preferably 3-18 carbon atoms. R is advantageously aliphatic, for example an alkyl group, preferably C₁-C₁₂alkyl. Most preferably, the organic hydroperoxide is tert-butyl-hydroperoxide or cumyl hydroperoxide.

The preferred amount of further catalyst is from about 0.0001 to 0.5, especially 0.0005 to 0.1 molar equivalent per mole of nitroxyl moiety, with a ratio of 0.001 to 0.05 moles of further catalyst per mole of nitroxyl moiety being the most preferred.

The reaction is preferably run at 0° to 100° C.; more preferably at 20° to 100° C., especially in the range from 20 to 80° C.

The C₆-C₁₈alk-1-ene may serve two functions both as reactant and as solvent for the reaction. The reaction can also be carried out using an inert organic or inorganic solvent.

Such solvent may be used, especially if the further catalyst is not very soluble in the C₅-C₁₈alk-1-ene. Typical inert solvents are acetonitrile, aromatic hydrocarbons like benzene, chlorobenzene, CCl₄, alcohols (e.g. methanol, ethanol, ethylene glycol, ethylene glycol monomethyl ether), or alkanes like hexane, decane etc., or mixtures thereof. Inorganic solvents such as water are possible as well.

The instant process can be run in air or in an inert atmosphere such as nitrogen or argon.

The instant process can be run under atmospheric pressure as well as under reduced or elevated pressure.

There are several variations of the instant process. One variation involves the addition of a solution of organic hydroperoxide to a mixture of the N-oxyl hindered amine, the C₅-C₁₈alk-1-ene and solvent (if used), and optionally further catalyst which has been brought to the desired temperature for reaction. The proper temperature may be maintained by controlling the rate of peroxide addition and/or by using a heating or cooling bath. After the hydroperoxide is added, the reaction mixture is conveniently stirred till the starting amineoxide has disappeared or is no longer being converted to the desired product, e.g. compound of formulae (A) to (O). The reaction can be monitored by methods known in the art such as UV-VIS spectroscopy, thin layer chromatography, gas chromatography or liquid chromatography. Additional portions of catalyst can be added while the reaction is in progress. After the initial hydroperoxide charge has been added to the reaction mixture, more hydroperoxide can be added dropwise to bring the reaction to completion.

A second variation of the instant process is to simultaneously add separate solutions of the hydroperoxide and the nitroxyl compound to a mixture of the C₅-C₁₈alk-1-ene, solvent (if used) and optionally further catalyst. The nitroxyl compound may be dissolved in water or the solvent used in the reaction, for example an alcohol. Some of the nitroxyl compound may be introduced into the reaction mixture prior to starting the peroxide addition, and all of the nitroxyl compound should be added prior to completing the peroxide addition.

Another variation of the instant process involves the simultaneous addition of separate solutions of the hydroperoxide and of the aqueous or solvent solution of the further catalyst to a mixture of the nitroxyl compound, C₅-C₁₈alk-1-ene, and solvent (if used). Some of the further catalyst may be introduced into the reaction mixture prior to starting the peroxide addition.

Still another variation of the instant process is the simultaneous addition of separate solutions of the hydroperoxide, of the aqueous or solvent solution of the nitroxyl compound, and of an aqueous or solvent solution of the further catalyst to the C₅-C₁₈alk-1-ene and solvent (if used). A portion of the nitroxyl compound and/or catalyst may be introduced into the reaction mixture prior to starting the hydroperoxide addition. All of the nitroxyl compound should be added prior to completing the hydroperoxide addition.

At the end of the reaction, the residual hydroperoxide may be carefully decomposed prior to the isolation of any products.

The present invention also pertains to a process, wherein the sterically hindered amine ether obtained by reacting a corresponding sterically hindered aminoxide with a C₅-C₁₈alk-1-ene in the presence of an organic hydroperoxide is subsequently hydrogenated. Hydrogenation may take place without premature isolation of the intermediate, without washings to destroy (excess) peroxide and without solvent change, excess olefin thereby being co-hydrogenated.

Advantageously, the hydrogenation is carried out in the presence of a hydrogenation catalyst.

The hydrogenation catalyst is preferably selected from the group consisting of platinum, palladium, ruthenium, rhodium, Lindlar catalyst, platinum compounds, palladium compounds, ruthenium compounds, rhodium compounds, iridium compounds, nickel compounds, zinc compounds and cobalt compounds.

The hydrogenation catalyst can be bound to an organic or inorganic polymer backbone, rendering a homogenous or heterogeneous catalytic system. Hydrogenation can also be carried out as transfer hydrogenation such as described in S. Murashi et al., Chem. Rev. (1998), 98, 2599-2660 or with further hydrogenation methods such as described in Larock, comprehensive organic transformations.

More preferably, the hydrogenation catalyst is selected from the group consisting of platinum, palladium, ruthenium, platinum compounds, palladium compounds and ruthenium compounds.

Most preferably, the hydrogenation catalyst is selected from the group consisting of platinum, palladium and ruthenium; platinum, palladium and ruthenium immobilized on carbon; PtO₂, Pd—CaCO₃—PbO, RuClH[PPh₃]₃, RhCl[PPh₃]₃ and RuH₂[P(Ph)₃]₄.

The preferred amount of hydrogenation catalyst is 0.0001-0.2 mol per mol of unsaturated amine ether moiety. The hydrogenation reaction is preferably run at 0 to 80° C.; especially in the range 20-60° C. The hydrogen pressure is preferably 1-20 atm.

The process for the preparation of a sterically hindered amine ether which comprises reacting a corresponding sterically hindered aminoxide with a C₅-C₁₈alk-1-ene in the presence of an organic hydroperoxide and optionally hydrogenating the reaction product results in a mixture of sterically hindered amine ethers. Hence, the instant invention relates also to mixtures of sterically hindered amine ethers defined below.

For example, the first mixture according to the instant invention is of formula (Za) 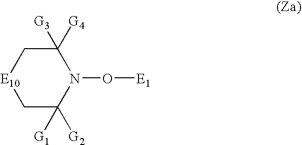
wherein G₁, G₂, G₃ and G₄ are as defined for formula (II);
E₁₀ is as defined for formula (IIa) and
E₁ is a mixture of the radicals 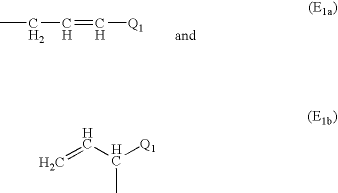
wherein Q₁ is C₂-C₁₅ alkyl;
with the proviso that the mixture of sterically hindered amine ethers is not a mixture of compounds of formula (100) and (101)

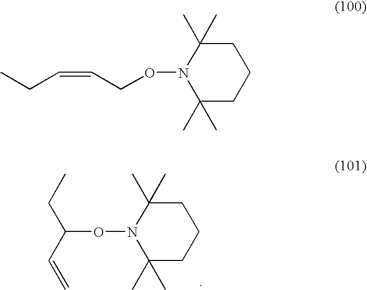

For instance, Q₁ is C₃-C₁₅alkyl.

For example, E₁₀ is a carbon atom which is substituted by —OH, ═O or by one or two organic residues containing in total 1-500 carbon atoms, especially E₁₀ is a carbon atom which is substituted by ═O or by one or two organic residues containing in total 1-500 carbon atoms.

Preferably, such mixture is represented by formulae (A) to (O), wherein each E is replaced by E₁.

Most preferably, such mixture is represented by formula (C), wherein each E is replaced by E₁.

Of further interest is the first mixture according to the present invention that consists of compounds containing groups of formula (Za).

The second mixture according to the instant invention contains at least one group of formula 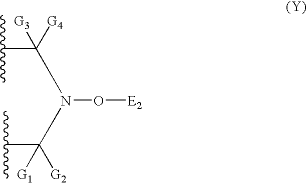
wherein G₁, G₂, G₃ and G₄ are as defined for formula (II) and
E₂ is a mixture of the radicals 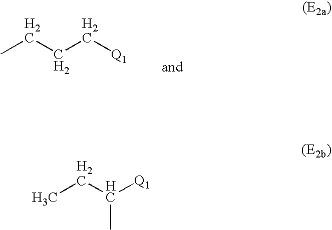
wherein Q₁ is C₂-C₁₅ alkyl.

Advantageously, such mixture is of formula (Ya) 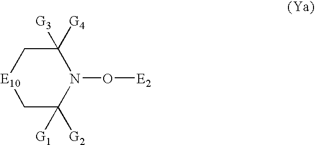
wherein G₁, G₂, G₃ and G₄ are as defined for formula (II);
E₁₀ is as defined for formula (IIa) and E₂ is as defined for formula (Y).

Preferably, such mixture is represented by formulae (A) to (O), wherein each E is replaced by E₂.

Most preferably, such mixture is represented by formula (C), wherein each E is replaced by E₂

Of further interest is the second mixture according to the present invention that consists of compounds containing groups of formula (Y).

A mixture of sterically hindered amine ethers is preferred, wherein the ratio E₁ₐ:E_{1b} and E₂ₐ:E_{2b} respectively is independently from 1:9 to 7:3, in particular from 1:4 to 3:2, for example 3:7 to 1:1, most preferred from 7:13 to 9:11.

For molecules containing more than one amine ether moiety, the groups E₁ₐ and E_{1b} and independently E₂ₐ and E_{2b} are distributed statistically in the molecule. The ratio E₁ₐ:E_{1b} and E₂ₐ:E_{2b} respectively in mixtures of the present invention is independent of the number of amine ether groups per molecule.

Further aspects of the present invention are

(i) mixtures obtainable by the process which comprises reacting a sterically hindered aminoxide with a C₅-C₁₈alk-1-ene in the presence of an organic hydroperoxide as well as

(ii) mixtures obtainable by hydrogenating a mixture according to (i).

Any mixture resulting from the processes of this invention may be separated to reveal the single amine ether components which can be afforded by conventional methods such as for example chromatography, distillation, precipitation or fractioned crystallization. However, for practical purposes it is not necessary to do so or it is even advantageous to use the mixtures unseparated.

Therefore, another aspect of the instant invention is the use of the mixtures according to this invention as stabilizers for organic material against degradation by light, oxygen and/or heat or as flame retardant for organic material, as well as a process for flame retarding or stabilizing an organic material against degradation by light, oxygen and/or heat, which process comprises applying to or incorporating into said material a mixture of sterically hindered amine ethers containing at least one group of formula (Y) or a mixture of sterically hindered amine ethers of formula (Za).

This invention further pertains to

a composition comprising

A) an organic material which is sensitive to oxidative, thermal and/or actinic degradation, and

B) at least one mixture of sterically hindered amine ethers containing at least one group of formula (Y) or at least one mixture of sterically hindered amine ethers of formula (Za).

Organic materials to be protected against the damaging effect of light, oxygen and/or heat, or against fire are in particular organic polymers, preferably synthetic organic polymers. The sterically hindered amine ether mixtures of this invention exhibit high thermal stability, compatibility and good persistence in the materials they are incorporated in or applied to.

Examples of polymers which can be protected in this way are the following:

1. Polymers of monoolefins and diolefins, for example polypropylene, polyisobutylene, polybut-1-ene, poly-4-methylpent-1-ene, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).
   2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).
   3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, propylene/butadiene copolymers, isobutylene/isoprene copolymers, ethylene/alkyl acrylate copolymers, ethylene/alkyl methacrylate copolymers, ethylene/vinyl acetate copolymers and their copolymers with carbon monoxide or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers andl mixtures thereof with other polymers, for example polyamides.
   4. Hydrocarbon resins (for example C₅-C₈) including hydrogenated modifications thereof (e.g. tackifiers) and mixtures of polyalkylenes and starch.
   5. Polystyrene, poly(p-methylstyrene), poly(α-methylstyrene).
   6. Copolymers of styrene or α-methylstyrene with dienes or acrylic derivatives, for example styrene/butadiene, styrene/acrylonitrile, styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.
   7. Graft copolymers of styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleimide on polybutadiene; styrene and maleimide on polybutadiene; styrene and alkyl acrylates or methacrylates on polybutadiene; styrene and acrylonitrile on ethylene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, MBS, ASA or AES polymers.
   8. Halogen-containing polymers such as polychloroprene, chlorinated rubbers, chlorinated and brominated copolymer of isobutylene-isoprene (halobutyl rubber), chlorinated or sulfochlorinated polyethylene, copolymers of ethylene and chlorinated ethylene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.
   9. Polymers derived from α,β-unsaturated acids and derivatives thereof such as polyacrylates and polymethacrylates; polymethyl methacrylates, polyacrylamides and polyacrylonitriles, impact-modified with butyl acrylate.
   10. Copolymers of the monomers mentioned under 9) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.
   11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.
   12. Homopolymers and copolymers of cyclic ethers such as polyalkylene glycols, polyethylene oxide, polypropylene oxide or copolymers thereof with bisglycidyl ethers.
   13. Polyacetals such as polyoxymethylene and those polyoxymethylenes which contain ethylene oxide as a comonomer; polyacetals modified with thermoplastic polyurethanes, acrylates or MBS.
   14. Polyphenylene oxides and sulfides, and mixtures of polyphenylene oxides with styrene polymers or polyamides.
   15. Polyurethanes derived from hydroxyl-terminated polyethers, polyesters or polybutadienes on the one hand and aliphatic or aromatic polyisocyanates on the other, as well as precursors thereof.
   16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 4/6, 12/12, polyamide 11, polyamide 12, aromatic polyamides starting from m-xylene diamine and adipic acid; polyamides prepared from hexamethylenediamine and isophthalic or/and terephthalic acid and with or without an elastomer as modifier, for example poly-2,4,4,-trimethylhexamethylene terephthalamide or poly-m-phenylene isophthalamide; and also block copolymers of the aforementioned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).
   17. Polyureas, polyimides, polyamide-imides, polyetherimids, polyesterimids, polyhydantoins and polybenzimidazoles.
   18. Polyesters derived from dicarboxylic acids and diols and/or from hydroxycarboxylic acids or the corresponding lactones, for example polyethylene terephthalate, polybutylene terephthalate, poly-1,4-dimethylolcyclohexane terephthalate and polyhydroxybenzoates, as well as block copolyether esters derived from hydroxyl-terminated polyethers; and also polyesters modified with polycarbonates or MBS.
   19. Polycarbonates and polyester carbonates.
   20. Polysulfones, polyether sulfones and polyether ketones.
   21. Crosslinked polymers derived from aldehydes on the one hand and phenols, ureas and melamines on the other hand, such as phenol/formaldehyde resins, urea/formaldehyde resins and melamine/formaldehyde resins.
   22. Drying and non-drying alkyd resins.
   23. Unsaturated polyester resins derived from copolyesters of saturated and unsaturated dicarboxylic acids with polyhydric alcohols and vinyl compounds as crosslinking agents, and also halogen-containing modifications thereof of low flammability.
   24. Crosslinkable acrylic resins derived from substituted acrylates, for example epoxy acrylates, urethane acrylates or polyester acrylates.
   25. Alkyd resins, polyester resins and acrylate resins crosslinked with melamine resins, urea resins, isocyanates, isocyanurates, polyisocyanates or epoxy resins.
   26. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.
   27. Blends of the aforementioned polymers (polyblends), for example PP/EPDM, Polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

Of particular interest is the use of mixtures of sterically hindered amine ethers of this invention, preferably sterically hindered amine ethers of formulae (A) to (O), wherein E is replaced by E₁ as defined for formula (Za) or by E₂ as defined for formula (Y), as stabilizers in synthetic organic polymers, for example a coating or a bulk polymer or article formed therefrom, especially in thermoplastic polymers and corresponding compositions as well as in coating compositions, for example in acid or metal catalyzed coating compositions. Thermoplastic polymers of most importance in present compositions are polyolefines (TPO) and their copolymers, such as listed above under items 1-3, thermoplastic polyurethane (TPU), thermoplastic rubber (TPR), polycarbonate, such as in item 19 above, and blends, such as in item 27 above. Of utmost importance are polyethylene (PE), polypropylene (PP), polycarbonate (PC) and polycarbonate blends such as PC/ABS blends.

In general the mixtures of sterically hindered amine ethers of present invention are added to the material to be stabilized in amounts of from 0.01 to 10%, preferably from 0.01 to 5%, in particular from 0.01 to 2% (based on the material to be stabilized). Particular preference is given to the use of the novel mixtures of sterically hindered amine ethers in amounts of from 0.05 to 1.5%, especially from 0.1 to 0.5%. Where mixtures of sterically hindered amine ethers of present invention are used as flame retardants, dosages are usually higher, e.g. 0.1 to 25% by weight, mainly 0.1 to 10% by weight of the organic material to be stabilized and protected against inflammation.

Incorporation into the materials can be effected, for example, by mixing in or applying the mixtures of sterically hindered amine ethers and, if desired, further additives by the methods which are customary in the art. Where polymers are involved, especially synthetic polymers, incorporation can take place prior to or during the shaping operation, or by applying the dissolved or dispersed compound to the polymer, with or without subsequent evaporation of the solvent. In the case of elastomers, these can also be stabilized as lattices. A further possibility for incorporating the mixtures of sterically hindered amine ethers into polymers is to add them before, during or directly after the polymerization of the corresponding monomers or prior to crosslinking. In this context the mixtures of sterically hindered amine ethers can be added as it is or else in encapsulated form (for example in waxes, oils or polymers).

The mixtures of sterically hindered amine ethers can also be added in the form of a masterbatch containing said compound in a concentration, for example, of from 2.5 to 25% by weight to the polymers that are to be stabilized.

The mixtures of sterically hindered amine ethers can judiciously be incorporated by the following methods:
as emulsion or dispersion (e.g. to lattices or emulsion polymers),
as a dry mixture during the mixing in of additional components or polymer mixtures,
by direct introduction into the processing apparatus (e.g. extruders, internal mixers, etc),
as solution or melt.

Novel polymer compositions can be employed in various forms and/or processed to give various products, for example as (to give) films, fibres, tapes, moulding compositions, profiles, or as binders for coating materials, adhesives or putties.

For example, the present compositions comprise as component C further additives.

For instance, the further additives are antioxidants, UV-absorbers, light stabilizers, metal deactivators, phosphites, phosphonites, hydroxylamines, nitrones, thiosynergists, peroxide scavengers, basic co-stabilizers, nucleating agents, fillers, reinforcing agents, benzofuranones, indolinones and/or flameproofing agents.

In addition to the mixtures of sterically hindered amine ethers, the novel compositions may as additional component C comprise one or more conventional additives such as, for example, those indicated below.

1. Antioxidants

1.1. Alkylated monophenols, for example 2, 6-di-tert-butyl-4-methylphenol, 2-tert-butyl-4,6-dimethylphenol, 2,6-di-ert-butyl-4-ethylphenol, 2,6-di-tert-butyl-4-n-butylphenol, 2,6-di-tert-butyl-4-isobutylphenol, 2,6-dicyclopentyl-4-methylphenol, 2-(o-methylcyclohexyl)-4,6-dimethylphenol, 2,6-dioctadecyl-4-methyl phenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof.

1.2. Alkylthiomethylphenols, for example 2, 4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonylphenol.

1.3. Hydroquinones and alkylated hydroquinones, for example 2, 6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis-(3,5-di-tert-butyl-4-hydroxyphenyl)adipate.

1.4. Tocopherols, for example α-tocopherol, β-tocopherol, γ-tocopherol, δ-tocopherol and mixtures thereof (Vitamin E).

1.5. Hydroxylated thiodiphenyl ethers, for example 2, 2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis-(3,6-di-sec-amylphenol), 4,4′-bis(2,6-dimethyl-4-hydroxyphenyl)disulfide.

1.6. Alkylidenebisphenols, for example 2, 2′-methylenebis(6-tert-butyl-4-methylphenol), 2,2′-methylenebis(6-tert-butyl-4-ethylphenol), 2,2′-methylenebis[4-methyl-6-(o-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(cc-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis-(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra-(5-tert-butyl-4-hydroxy-2-methylphenyl)pentane.

1.7. O-, N- and S-benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate.

1.8. Hydroxybenzylated malonates, for example dioctadecyl-2,2-bis-(3,5-di-tert-butyl-2-hydroxybenzyl)-malonate, di-octadecyl-2-(3-tert-butyl-4-hydroxy-5-methylbenzyl)-malonate, di-dodecylmercaptoethyl-2,2-bis-(3,5-di-tert-butyl-4-hydroxybenzyl)malonate, bis[4-(1,1,3,3-tetrafnethylbutyl)phenyl]-2,2-bis(3,5-di-tert-butyl-4-hydroxybenzyl)malonate.

1.9. Aromatic hydroxybenzyl compounds, for example 1, 3,5-tris-(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol.

1.10. Triazine Compounds, for example 2, 4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris-(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-triazine, 1,3,5-tris(3,5-dicyclohexyl-4-hydroxybenzyl)isocyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxylauranilide, 4-hydroxystearanilide, octyl N-(3,5-di-tert-butyl-4-hydroxyphenyl)carbamate.

1.13. Esters of 5-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.14. Esters of 5-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.15. Esters of 53-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1 supplied by Uniroyal).

1.18. Ascorbic acid (vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenlenediamine, 4-(p-toluenesulfamoyl)diphenylamine, N,N′-dimethyl-N,N′-di-sec-butyl-p-phenylenediamine, diphenylamine, N-allyidiphenylamine, 4-isopropoxy-diphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyidiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono-und dialkylated tert-butyldiphenylamines, 2,3-dihydro-3,3-dimethyl-4H-1,4-benzothiazine, phenothiazine, a mixture of mono-und dialkylated tert-butyl/tert-octylphenothiazines, a mixture of mono-und dialkylated tert-octyl-phenothiazines, N-allylphenothiazin, N,N,N′,N′-tetraphenyl-1,4-diaminobut-2-ene, N,N-bis-(2,2,6,6-tetramethyl-piperid-4-yl-hexamethylenediamine, bis(2,2,6,6-tetramethylpiperid-4-yl)-sebacate, 2,2,6,6-tetramethylpiperidin-4-one, 2,2,6,6-tetramethylpiperidin-4-ol.

2. UV Absorbers and Light Stabilisers

2.1. 2-(2′-Hydroxyphenyl)benzotriazoles, for example 2-(2′-hydroxy-5′-methylphenyl)-benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis-(α,α-dimethylbenzyl)-2′-hydroxyphenyl)benzotriazole, 2-(3′-tert-butyl-2′-hydroxy-5′-(2-octyloxycarbonylethyl)phenyl)-5-chloro-benzotriazole, 2-(3′-tert-butyl-5′-[2-(2-ethylhexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylene-bis-[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH₂CH₂—COO—CH₂CH₂]—₂ where R=3′-tert-butyl-4′-hydroxy-5′-2H-benzotriazol-2-ylphenyl, 2-[2′-hydroxy-3′-(α,α-dimethylbenzyl)-5′-(1,1,3,3-tetramethylbutyl)-phenyl]benzotriazole; 2-[2′-hydroxy-3′-(1, 1,3,3-tetramethylbutyl)-5′-(α,α-dimethylbenzyl)-phenyl]benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

2.3. Esters of substituted and unsubstituted benzoic acids, as for example 4-tertbutyl-phenyl salicylate, phenyl salicylate, octylphenyl salicylate, dibenzoyl resorcinol, bis(4-tert-butylbenzoyl)resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2.4. Acrylates, for example ethyl α-cyano-β,β-diphenylacrylate, isooctyl α-cyano-β,β-diphenylacrylate, methyl α-carbomethoxycinnamate, methyl α-cyano-β-methyl-p-methoxy-cinnamate, butyl α-cyano-β-methyl-p-methoxy-cinnamate, methyl α-carbomethoxy-p-methoxycinnamate and N-(β-carbomethoxy-β-cyanovinyl)-2-methylindoline.

2.5. Nickel compounds, for example nickel complexes of 2,2′-thio-bis-[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyldithiocarbamate, nickel salts of the monoalkyl esters, e.g. the methyl or ethyl ester, of 4-hydroxy-3,5-di-tertbutylbenzylphosphonic acid, nickel complexes of ketoximes, e.g. of 2-hydroxy-4-methylphenyl undecylketoxime, nickel complexes of 1-phenyl-4-lauroyl-5-hydroxypyrazole, with or without additional ligands.

2.6. Further sterically hindered amines, for example bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl)n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butane-tetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl)malonate, 3-n-octyl-7,7,9,9-tetramethyl-1,3,8-triazaspiro[4.5]decan-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′-bis-(2,2,6,6-tetramethyl-4-piperidyl)hexamethylenediamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)ethane, the condensate of 2-chloro-4,6-di-(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidin-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensation product of N,N′-bis(2,2,6,6-tetramethyl-4-piperidyl)hexamethylenediamine and 4-cyclohexylamino-2,6-dichloro-1,3,5-triazine, a condensation product of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); N-(2,2,6,6-tetramethyl-4-piperidyl)-n-dodecylsuccinimid, N-(1,2,2,6,6-pentamethyl-4-piperidyl)-n-dodecylsuccinimid, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro [4,5]decane und epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexamethylenediamine, diester of 4-methoxy-methylene-malonic acid with 1,2,2,6,6-pentamethyl-4-hydroxypiperidine, poly[methylpropyl-3-oxy-4-(2,2,6,6-tetramethyl-4-piperidyl)]siloxane, reaction product of maleic acid anhydride-α-olefin-copolymer with 2,2,6,6-tetramethyl-4-aminopiperidine or 1,2,2,6,6-pentamethyl-4-aminopiperidine, 2,4-bis[N-(1-cyclohexyloxy-2,2,6,6-tetramethylpiperidine-4-yl)-N-butyl-amino]-6-(2-hydroxyethyl)amino-1,3,5-triazine.

2.7. Oxamides, for example 4, 4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.8. 2-(2-Hydroxyphenyl)-1,3,5-triazines, for example 2, 4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxy-propoxy)phenyl]4,6-bis(2,4-dimethyl)-1,3,5-triazine, 2-[2-hydroxy-4-(2-hydroxy-3-octyloxy-propyloxy)phenyl]-4,6-bis(2,4-dimethyl)-1,3,5-triazine, 2-[4-(dodecyloxy/tridecyloxy-2-hydroxypropoxy)-2-hydroxy-phenyl]-4,6-bis(2,4-dimethylphenyl)-1,3,5-triazine, 2-[2-hydroxy-4-(2-hydroxy-3-dodecyloxy-propoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxy-propoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine, 2-{2-hydroxy-4-[1-octyloxycarbonyl-ethoxy]phenyl}-4,6-bis(4-phenylphenyl)-1,3,5-triazine wherein the octyl moiety is a mixture of different isomers.

3. Metal deactivators, for example N,N′-diphenyloxamide, N-salicyloyl-N′-salicyloyl hydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyl dihydrazide, oxanilide, isophthaloyl dihydrazide, sebacoyl bisphenylhydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenyl alkyl phosphites, phenyl dialkyl phosphites, tris(nonylphenyl)phosphite, trilauryl phosphite, trioctadecyl phosphite, distearyl pentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,6-di-tert-butyl-4-methylphenyl)-penteerythritol diphosphite, diisodecyloxypentaerythritol diphosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-12H-di-benz[d,g]-1,3,2-dioxaphosphoci n, 6-fluoro-2,4,8,10-tetra-tert-butyl-12-methyl-dibenz[d,g]-1,3,2-dioxaphosphocin, bis(2,4-di-tert-butyl-6-methylphenyl)methyl phosphite, bis(2,4-di-tert-butyl-6-methylphenyl)ethyl plhosphite, 2,2′,2″-nitrilo[triethyltris(3,3′,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-di-yl)phosphite.

Especially preferred are the following phosphites:

Tris(2,4-di-tert-butylphenyl)phosphite (Irgafos®168, Ciba-Geigy), tris(nonylphenyl)phosphite,

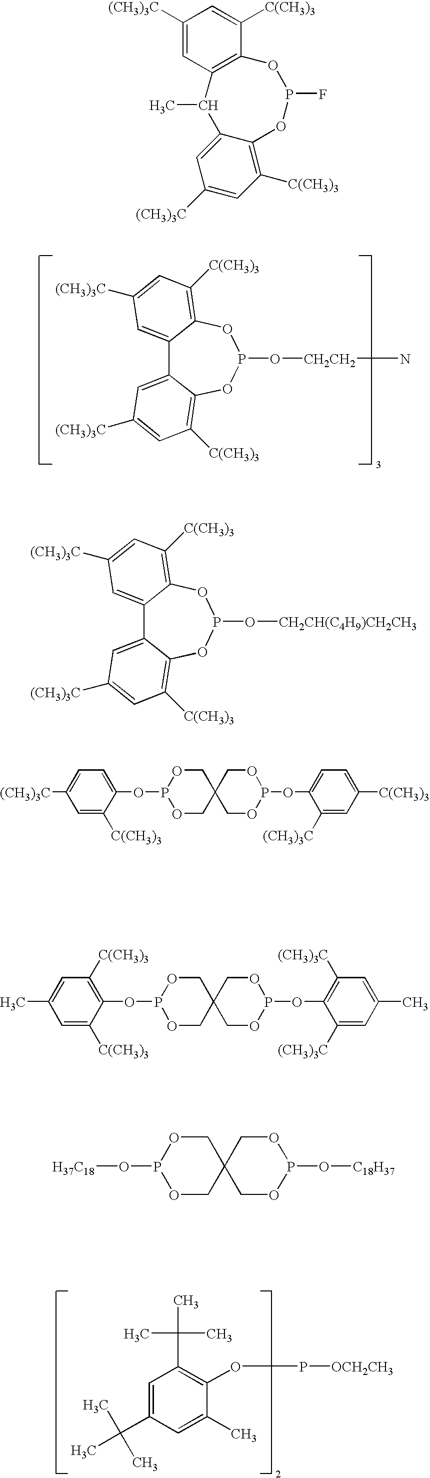

5. Hydroxylamines, for example, N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N,N-dialkylhydroxylamine derived from hydrogenated tallow amine.

6. Nitrones, for example, N-benzyl-alpha-phenyl-nitrone, N-ethyl-alpha-methyl-nitrone, N-octyl-alpha-heptyl-nitrone, N-lauryl-alpha-undecyl-nitrone, N-tetradecyl-alpha-tridecyl-nitrone, N-hexadecyl-alpha-pentadecyl-nitrone, N-octadecyl-alpha-heptadecyl-nitrone, N-hexadecyl-alpha-heptadecyl-nitrone, N-ocatadecyl-alpha-pentaclecyl-nitrone, N-heptadecyl-alpha-heptadecyl-nitrone, N-octadecyl-alpha-hexadecyl-nitrone, nitrone derived from N,N-dialkylhydroxylamine derived from hydrogenated tallow amine.

7. Thiosynergists, for example, dilauryl thiodipropionate or distearyl thiodipropionate.

8. Peroxide scavengers, for example esters of β-thiodipropionic acid, for example the lauryl, stearyl, myristyl or tridecyl esters, mercaptobenzimidazole or the zinc salt of 2-mercaptobenzimidazole, zinc dibutyldithiocarbamate, dioctadecyl disulfide, pentaerythritol tetrakis(β-dodecylmercapto)propionate.

9. Polyamide stabilisers, for example, copper salts in combination with iodides and/or phosphorus compounds and salts of divalent manganese.

10. Basic co-stabilisers, for example, melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids for example calcium stearate, zinc stearate, magnesium behenate, magnesium stearate, sodium ricinoleate and potassium palmitate, antimony pyrocatecholate or zink pyrocatecholate.

11. Nucleating agents, for example, inorganic substances such as talcum, metal oxides such as titanium dioxide or magnesium oxide, phosphates, carbonates or sulfates of, preferably, alkaline earth metals; organic compounds such as mono- or polycarboxylic acids and the salts thereof, e.g. 4-tert-butylbenzoic acid, adipic acid, diphenylacetic acid, sodium succinate or sodium benzoate; polymeric compounds such as ionic copolymers (ionomers).

12. Fillers and reinforcing agents, for example, calcium carbonate, silicates, glass fibres, glass bulbs, asbestos, talc, kaolin, mica, barium sulfate, metal oxides and hydroxides, carbon black, graphite, wood flour and flours or fibers of other natural products, synthetic fibers.

13. Other additives, for example, plasticisers, lubricants, emulsifiers, pigments, rheology additives, catalysts, flow-control agents, optical brighteners, flameproofing agents, antistatic agents and blowing agents.

14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A-4316622; DE-A-4316876; EP-A-0589839 or EP-A-0591102 or 3-[4-(2-acetoxyethoxy)phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, 3-(2,3-di-methylphenyl)-5,7-di-tert-butyl-benzofuran-2-one.

The conventional additives are judiciously employed in amounts of 0.1-10% by weight, for example 0.2-5% by weight, based on the material to be stabilized.

The following examples are for illustrative purposes only and are net to be construed to limit the instant invention in any manner whatsoever. Percentages given are usually percent by weight if not otherwise indicated.

Abbreviations used:

Bu butyl

DEPT distortionless enhancement by polarization transfer

DSC differential scanning calorimetry

HSQC heteronuclear single quantum coherence

NMR nuclear magnetic resonance

TEMPO 2,2,6,6-tetramethylpiperidine-N-oxide

Coupling of nitroxides with 1-octene followed by hydrogenation versus coupling of nitroxides with octane (Examples 1-7)

### EXAMPLE 1

### Preparation of an O-octenyl Sterically Hindered Amine Ether from the Corresponding Nitroxide and 1-n-octene with a tert-BuOOH/CuBr₂ Catalyst System

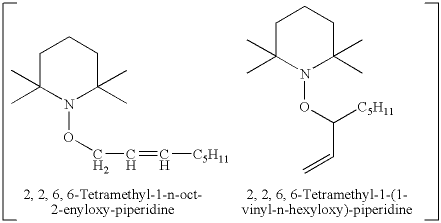

To a stirred mixture of 7 g (45 mmol) TEMPO, 52.1 g (450 mmol) 1-n-octene and 0.1 g (0.45 mmol) CuBr₂ are added at 60° C. within 60 minutes 17.4 g (135 mmol) tert-butyl-hydroperoxide (70% aqueous solution). The colorless reaction mixture is cooled down to 25° C. and stirred with 85 g of an aqueous 20% Na₂SO₃ solution until the disappearance of excess tert-butyl-hydroperoxide. The aqueous phase is then separated and washed with cyclohexane. The combined organic phases are washed with brine, dried over MgSO₄, filtered and the solvent is distilled off on a rotary-evaporator. Purification by flash-chromatography (silica gel, hexane/ethylacetate 9/1) affords 9.8 g (81% of theory) of a mixture of 2,2,6,6-tetramethyl-1-n-oct-2-enyloxy-piperidine (ca 40 mol % by ¹H-NMR) and 2,2,6,6-tetramethyl-1-(1-vinyl-n-hexyloxy)-piperidine (ca 60 mol % by ¹H-NMR).

Analysis required for C₁₇H₃₃NO (267.45): C 76.34%, H 12.44%, N 5.24%; found: C 75.01%, H 12.27%, N 4.85%.

¹H-NMR (CDCl₃), δ (ppm, O—C(n) Hₓ only): 4.06 (q-like, O—C(3)H), 4.21 and 4.33 (d-like, O—C(1)H₂).

¹³C(DEPT)-NMR (CDCl₃), δ (ppm, O—CHₓ and ═CH_{y} only): 73.3 (O—CH₂), 78.4 (O—CH₂), 85.8 (O—CH), 115.3 (═CH₂), 125.2 (═CH), 125.5 (═CH), 132.9 (═CH), 133.9 (═CH), 141.2 (═CH).

### EXAMPLE 2

### Preparation of an O-octenyl Sterically Hindered Amine Ether (Same Compound as in Example 1) from the Corresponding Nitroxide and 1-n-octene with a tert-BuOOH/Bu₄NI Catalyst System

To a stirred mixture of 7.8 g (50 mmol) TEMPO, 56.1 g (500 mmol) 1-n-octene and 0-18 g (0.5 mmol) tetrabutylammoniumiodide are added at 55° C. within 50 minutes 6.4 g (50 mmol) tert-butylhydroperoxide (70% aqueous solution). The temperature is maintained at 55° C. for 50 minutes until all of the TEMPO is reacted. The reaction mixture is cooled down to 25° C. and stirred with 31 g of an aqueous 20% Na₂SO₃ solution until the disappearance of excess tert-butylhydroperoxide. The aqueous phase is then separated and washed with cyclohexane. The combined organic phases are passed through a plug of silica gel and washed with brine, dried over MgSO₄, filtered and the solvent is distilled off on a rotary-evaporator, yielding 11 g (82.3% of theory) of product exhibiting the same ¹H-NMR spectrum as above.

Analysis required for C₁₇H₃₃NO (267.45): C 76.34%, H 12.44%, N 5.24%; found: C 75.48%, H 12.30%, N 5.21%.

### EXAMPLE 3

### Preparation of an O-octyl Sterically Hindered Amine Ether by Hydrogenation of O-octenyl Sterically Hindered Amine Ether (Product of Example 1 or 2)

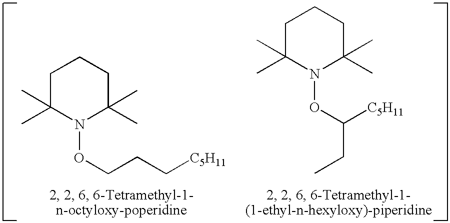

A mixture of 10 g (37.4 mmol) of the crude product of example 1 or 2 and 1.9 g Pd on charcoal (10%) in 100 ml methanol is hydrogenated at 25° C. and 4 bar hydrogen pressure. Filtration and evaporation of the solvent yields 7 g (69.5% of theory) of a slightly orange oil, a mixture of 2,2,6,6-tetramethyl-1-n-octyloxy-piperidine (ca 40 mol % by ¹H-NMR) and 1-(1-ethyl-n-hexyloxy)-2,2,6,6-tetramethyl-piperidine (ca 60 mol % by ¹H-NMR).

Analysis required for C₁₇H₃₅NO (269.48): C 75.77%, H 13.09%, N 5.20%; found: C 74.79%, H 12.72%, N 5.19%.

¹H-NMR (CDCl₃), δ (ppm, O—C(n)Hₓ only): 3.66 (p-like, O—C(3)H), 3.72 (t, J=6.7 Hz, O—C(1)H₂).

¹³C(DEPT)-NMR (CDCl₃), δ (ppm, O—C(n)Hₓ only): 76.94 (O—C(1)H₂), 83.12 (O—C(3)H).

### EXAMPLE 4 (COMPARISON)

### Preparation of an O-octyl Sterically Hindered Amine Ether by Direct Coupling of the Corresponding Nitroxide and n-octane with a tert-BuOOH/CuBr₂ Catalyst System

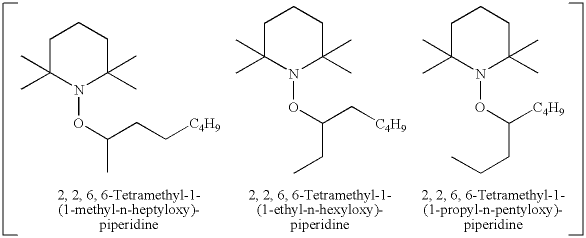

To a stirred mixture of 4.7 g (30 mmol) TEMPO, 34.3 g (300 mmol) n-octane and 0.067 g (0.30 mmol) CuBr₂ are added at 60° C. within 60 minutes 11.6 g (90 mmol) tert-butylhydroperoxide (70% aqueous solution). The temperature of the reaction mixture is maintained at 60° C. for 25 hours. To the still reddish solution are added another 0.30 mmol CuBr₂/90 mmol tert-butylhydroperoxide and the reaction mixture is stirred at 80° C. for 1.7 hours. The greenish emulsion is cooled down to 25° C. and stirred with 75 g of an aqueous 20% Na₂SO₃ solution until the disappearance of excess tert-butylhydroperoxide. The aqueous phase is then separated and washed with octane. The combined organic phases are washed with brine, dried over MgSO₄, filtered and the solvent is distilled off on a rotary-evaporator. Purification by flash-chromatography (silica gel, hexane/ethylacetate 9/1) affords 4.9 g (60% of theory) of a mixture of 2,2,6,6-tetramethyl-1-(1-methyl-n-heptyloxy)piperidine (ca 40 mol % by ¹H-NMR), 2,2,6,6-tetramethyl-1-(1-ethyl-n-hexyloxy)-piperidine (ca 30 mol % by ¹H-NMR) and 2,2,6,6-tetramethyl-1-(propyl-n-pentyloxy)-piperidine (ca 30 mol % by ¹H-NMR).

Analysis required for C₁₇H₃₅NO (269.47): C 75.77%, H 13.09%, N 5.20%; found: C 75.72%, H 13.06%, N 5.02%.

¹H-NMR (CDCl₃), δ (ppm, O—C(n)Hₓ only): 3.66 (m, O—C(3)H), 3.72 (m, O—C(4)H), 3.85 (m, O—C(2)H).

¹³C(DEPT)-NMR (CDCl₃), δ (ppm, O—C(n)Hₓ only): 78.43 (O—C(2)H), 81.75 (O—C(4)H), 83.12 (O—C(3)H).

¹³C/¹H-correlation is established by HSQC-spectroscopy. O—C(2)H and O—C(4)H are tentatively assigned according to the Grant-Paul rules describing the empiric calculation of ¹³C-chemical shifts.

### EXAMPLE 5

### Preparation of an O-octenyl Sterically Hindered Amine Ether from the Corresponding Nitroxide and 1-n-octene with a tert-BuOOH/CuBr₂ Catalyst System

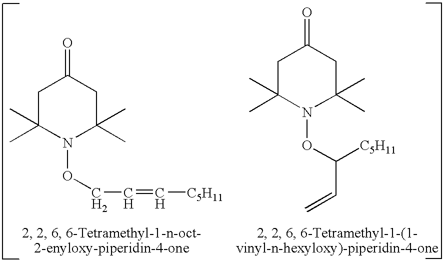

To a stirred mixture of 7.7 g (45 mmol) 2,2,6,6-tetramethyl-4-piperidon-N-oxide, 52.1 g (450 mmol) 1-n-octene and 0.1 g (0.45 mmol) CuBr₂ are added dropwise at 60° C. 17.4 g (135 mmol) tert-butylhydroperoxide (70% aqueous solution). The temperature of the reaction mixture is held at 60° C. for a total of 2.4 hours. The green emulsion is cooled down to 25° C. and stirred with 85 g of an aqueous 20% Na₂SO₃ solution until the disappearance of excess tert-butylhydroperoxide. The aqueous phase is then separated and washed with cyclohexane. The combined organic phases are washed with brine, dried over MgSO₄, filtered and the solvent is distilled off on a rotary-evaporator. Purification by flash-chromatography (silica gel, hexane/ethylacetate 9/1) affords 7 g (55% of theory) of a mixture of 2,2,6,6-tetramethyl-1-n-oct-2-enyloxy-piperidine-4-one (ca 40 mol % by ¹H-NMR) and 2,2,6,6-tetramethyl-1-(1-vinyl-n-hexyloxy)-piperidine-4-one (ca 60 mol % by ¹H-NMR).

Analysis required for C₁₇H₃₁NO₂ (281.44): C 72.55%, H 11.10%, N 4.98%; found: C 72.50%, H 10.84%, N 4.77%.

¹H-NMR (CDCl₃), δ (ppm, O—C(n)Hₓ only): 4.15 (q-like, O—C(3)H), 4.30 and 4.42 (d-like, O—C(1)H₂).

¹³C(DEPT)-NMR (CDCl₃), δ (ppm, O—CHₓ and ═CH_{y} only): 73.4 (O—CH₂), 78.5 (O—CH₂), 86.2 (O—CH), 116.5 (═CH₂), 124.3 (═CH), 124.8 (═CH), 133.7 (═CH), 134.8 (═CH), 140.4 (═CH).

### EXAMPLE 6

### Preparation of an O-octyl Sterically Hindered Amine Ether by Hydrogenation of an O-octenyl Sterically Hindered Amine Ether (Product of Example 5)

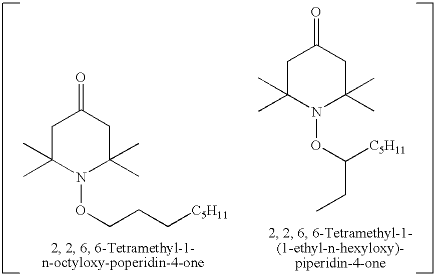

A mixture of 12.3 g (43.7 mmol) of the product of example 5 and 0.8 g Pt on charcoal (10%) in 120 ml ethyl acetate is hydrogenated at 25° C. and 4 bar hydrogen pressure. Filtration and evaporation of the solvent yields 9.15 g (73.9% of theory) of colorless oil consisting of a mixture of 2,2,6,6-tetramethyl-1-n-octyloxy-piperidin-4-one (ca 40 mol % by ¹H-NMR) and 1-(1-ethyl-n-hexyloxy)-2,2,6,6-tetramethyl-piperidin-4-one (ca 60 mol % by ¹H-NMR).

Analysis required for C₁₇H₃₃NO₂ (283.45): C 72.04%, H 11.73%, N 4.94%; found: C 71.65%, H 11.36%, N 4.86%.

¹H-NMR (CDCl₃), δ (ppm, O—C(n)Hₓ only): 3.73 (p-like, O—C(3)H), 3.82 (t, J=ca 6 Hz, O—C(1)H₂).

¹³C(DEPT)-NMR (CDCl₃), δ (ppm, O—C(n)Hₓ only): 77.20 (O—C(1)H₂), 83.62 (O—C(3)H).

### EXAMPLE 7 (COMPARISON)

### Preparation of an O-octyl Sterically Hindered Amine Ether by Direct Coupling of the Corresponding Nitroxide and n-octane with a tert-BuOOH/CuBr₂ Catalyst System

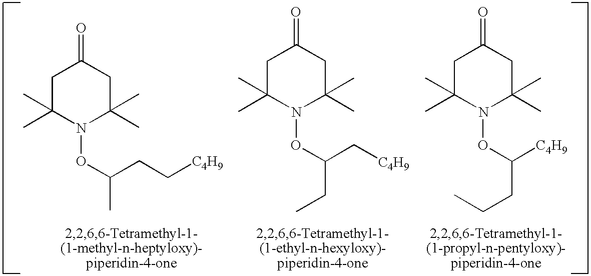

To a stirred mixture of 15.3 g (90 mmol) 2,2,6,6-tetramethyl-4-piperidon-N-oxide, 102.9 g (900 mmol) n-octane and 0.2 g (0.90 mmol) CuBr₂ are added at 80° C. within 60 minutes 34.8 g (270 mmol) tert-butylhydroperoxide (70% aqueous solution). The temperature of the reaction mixture is held at 80° C. for 1.5 hours. The green emulsion is cooled down to 25° C. and stirred with 170 g of an aqueous 20% Na₂SO₃ solution until the disappearance of excess tert-butylhydroperoxide. The aqueous phase is then separated and washed with cyclohexane. The combined organic phases are washed with brine, dried over MgSO₄, filtered and the solvent is distilled off on a rotary-evaporator. Purification by flash-chromatography (silica gel, hexane/ethylacetate 8.5/1.5) affords 7.5 g (29% of theory) of a mixture of 2,2,6,6-tetramethyl-1-(1-methyl-n-heptyloxy)-piperidin-4-one (ca 40 mol % by ¹H-NMR), 2,2,6,6-tetramethyl-1-(1-ethyl-n-hexyloxy)-piperidin-4-one (ca 30 mol % by ¹H-NMR) and 2,2,6,6-tetramethyl-1-(propyl-n-pentyloxy)-piperidin-4-one (ca 30 mol % by ¹H-NMR).

Analysis required for C₁₇H₃₃NO₂ (283.45): C 72.04%, H 11.73%, N 4.94%; found: C 71.80%, H 11.57%, N 4.68%.

¹H-NMR (CDCl₃), δ (ppm, O—C(n)Hₓ only): 3.75 (m, O—C(3)H), 3.81 (m, O—C(4)H), 3.95 (m, O—C(2)H).

¹³C(DEPT)-NMR (CDCl₃), δ (ppm, O—C(n) Hₙ only): 78.8 (O—C(2)H), 82.2 (O—C(4)H), 83.6 (O—C(3)H).

¹³C/1H-correlation is established by HSQC-spectroscopy. O—C(2)H and O—C(4)H are tentatively assigned according to the Grant-Paul rules describing the empiric calculation of ¹³C-chemical shifts.

### EXAMPLE 8

### Preparation of an O-octenyl Sterically Hindered Amine Ether by Coupling of the Corresponding Nitroxide and 1-n-octene with a tert-BuOOH/CuBr₂ Catalyst System

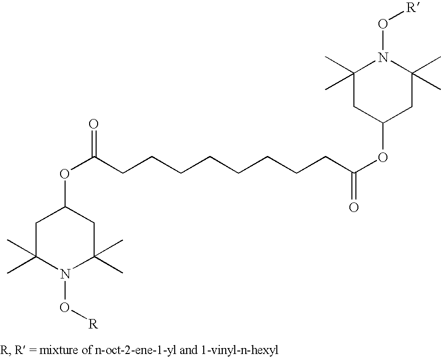

To a stirred mixture of 10.2 g (20 mmol) bis(1-oxyl-2,2,6,6-tetramethylpiperidine-4-yl)sebacate (Prostab® 5415, Ciba Specialty Chemicals Inc.), 46.3 g (400 mmol) 1-n-octene and 0.09 g (0.4 mmol) CuBr₂ are added at 60° C. within 30 minutes 7.7 g (60 mmol) tertbutylhydroperoxide (70% aqueous solution). The green emulsion is cooled down to 25° C. and stirred with 19 g of an aqueous 20% Na₂SO₃ solution until the disappearance of excess tert-butylhydroperoxide. The organic phase is separated, washed with brine, dried over MgSO₄, filtered and the solvent is distilled off on a rotary-evaporator. Purification by flash-chromatography (silica gel, hexane/ethylacetate 8.5/1.5) affords 8.6 g (59% of theory) of a slightly yellow oil. The ratio of the sum of R, R′=n-oct-2-ene-1-yl to R, R′=1-vinyl-n-hexyl is about 40 mol % to 60 mol % (by ¹H-NMR).

Analysis required for C₄₄H₈₀N₂O₆ (733.13): C 72.09%, H 11.00%, N 3.82%; found: C 71.24%, H 10.66%, N 3.60%.

¹H-NMR (CDCl₃), δ (ppm, O—C(n)Hₓ only): 4.06 (q-like, O—C(3)H), 4.21 and 4.32 (d-like, O—C(1)H₂).

¹³C(DEPT)-NMR (CDCl₃), δ (ppm, O—CHₓ and ═CH_{y} only): 73.4 (O—CH₂), 78.5 (O—CH₂), 85.9 (O—CH), 115.8 (═CH₂), 124.8 (═CH), 125.2 (═CH), 133.2 (═CH), 134.3 (═CH), 140.8 (═CH).

### EXAMPLE 9

### Preparation of an O-octenyl Sterically Hindered Amine Ether (same Compound as in Example 8) by Coupling of the Corresponding Nitroxide and 1-n-octene with a tert-BuOOH/Bu₄NI Catalyst System

To a stirred mixture of 12.8 g (25 mmol) bis(1-oxyl-2,2,6,6-tetramethylpiperidine-4-yl)sebacate (Prostab® 5415, Ciba Specialty Chemicals Inc.), 56.11 g (500 mmol) 1-n-octene and 0.185 g (0.5 mmol) tetrabutylammoniumiodide are added at 60° C. within 30 minutes 9.66 g (75 mmol) tert-butylhydroperoxide (70% aqueous solution). The temperature of the reaction mixture is held at 60° C. for 2 hours. The yellow emulsion is cooled down to 25° C. and stirred with 47 g of an aqueous 20% Na₂SO₃ solution until the disappearance of excess tertbutylhydroperoxide. The aqueous phase is then separated and washed with pentane. The combined organic phases are washed with brine, dried over MgSO₄, filtered and the solvent is distilled off on a rotary-evaporator to give 14.49 g (79% of theory) of a yellowish oil exhibiting the same ¹H-NMR spectrum as the mixture of example 8.

### EXAMPLE 10

### Preparation of an O-octyl Sterically Hindered Amine Ether by Hydrogenation of an O-octenyl Sterically Hindered Amine Ether (Product of Example 8)

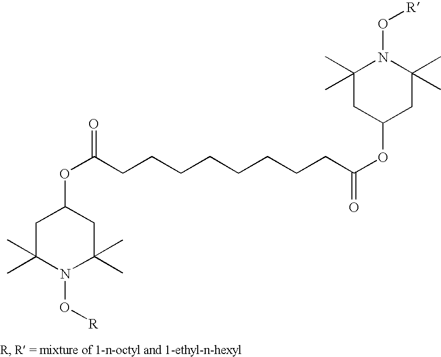

A mixture of 3.95 g (5.4 mmol) of the product from example 8 and 0.3 g Pd on charcoal (10%) in 50 ml hexane is hydrogenated at 50° C. and 4 bar hydrogen pressure. Filtration and evaporation of the solvent yields 3.3 g (83% of theory) of a slightly orange oil. The ratio of the sum of R, R′=1-n-octyl to R, R′=1-ethyl-n-hexyl is about 40 mol % to 60 mol % (by ¹H-NMR).

Analysis required for C₄₄H₈₄N₂O₆ (737.16): C 71.69%, H 11.49%, N 3.80%; found: C 70.68%, H 11.64%, N 3.70%.

¹H-NMR (CDCl₃), δ (ppm, O—C(n)Hₓ only): 3.67 (p-like, O—C(3)H), 3.72 (t, J=6.8 Hz, O—C(1)H₂).

¹³C(DEPT)-NMR (CDCl₃), δ (ppm, O—C(n)Hₓ only): 77.05 (O—C(1)H₂), 83.3 (O—C(3)H).

### EXAMPLE 11

### Preparation of an O-octyl Sterically Hindered Amine Ether by Coupling of the Corresponding Nitroxide and 1-n-octene with a tert-BuOOH/CuCl₂ Catalyst System Followed by Hydrogenation (Same Product as in Example 10)

Procedure using 1.6 eq tert-BuOOH. (Reducing the amount of tert-BuOOH reduces the amount of dialkylperoxides remaining in the product as revealed by DSC).

To a stirred mixture of 17.9 g (35 mmol) bis(1-oxyl-2,2,6,6-tetramethylpiperidine-4-yl)sebacate (Prostab® 5415, Ciba Specialty Chemicals Inc.), 81 g (700 mmol) 1-n-octene and 0.1 g (0.7 mmol) CuCl₂ are added at 60° C. within 30 minutes 7.2 g (56 mmol) tertbutylhydroperoxide (70% aqueous solution). The temperature of the reaction mixture is held at 60° C. for a total of 2.5 hours. The green emulsion is cooled down to 25° C. and stirred with 22 g of an aqueous 20% Na₂SO₃ solution until the disappearance of excess tert-butylhydroperoxide. The organic phase is separated, washed with brine, dried over MgSO₄, filtered and the solvent distilled off on a rotary-evaporator yielding a yellow oil.

After addition of 300 ml ethylacetate and 0.7 g 10% Pt on carbon (0.35 mmol) the mixture is hydrogenated at 25° C./4 bar hydrogen pressure. Filtration and evaporation of the solvent yields 23 g (89% of theory) of a very slightly brownish oil exhibiting the same ¹H-NMR spectrum as in example 10.

Analysis required for C₄₄H₈₄N₂O₆ (737.16): C 71.69%, H 11.49%, N 3.80%; found: C 70.99%, H 11.20%, N 3.80%.

### EXAMPLE 12

### Preparation of an O-octyl Sterically Hindered Amine Ether (Same Product as in Examples 10 and 11) by Coupling of the Corresponding Nitroxide and 1-n-octene with a tertBuOOH/Bu₄NI Catalyst System Followed by Hydrogenation

Procedure using 1.6 eq tert-BuOOH (Reducing the amount of tert-BuOOH reduces the amount of dialkylperoxides remaining in the product as revealed by DSC).

To a stirred mixture of 17.9 g (35 mmol) bis(1-oxyl-2,2,6,6-tetramethylpiperidine-4-yl)sebacate (Prostab® 5415, Ciba Specialty Chemicals Inc.), 81 g (700 mmol) 1-n-octene and 0.26 g (0.7 mmol) Bu₄NI are added at 60° C. within 30 minutes 7.2 g (56 mmol) tertbutylhydroperoxide (70% aqueous solution). The temperature of the reaction mixture is held at 60° C. for a total of 4.75 hours. After addition of another 0.9 g (7 mmol) tertbutylhydroperoxide and further stirring for 30 minutes the yellow emulsion is cooled down to 25° C. and stirred with 22 g of an aqueous 20% Na₂SO₃ solution until the disappearance of excess tert-butylhydroperoxide. The organic phase is separated, washed with brine, dried over MgSO₄, filtered and the solvent distilled off on a rotary-evaporator yielding a yellow oil.

After addition of 300 ml ethylacetate and 0.7 g 10% Pt on carbon (0.35 mmol) the mixture is hydrogenated at 25° C./4 bar hydrogen pressure. Filtration and evaporation of the solvent yields 23.9 g (93% of theory) of a very slightly yellowish oil exhibiting the same ¹H-NMR spectrum as in example 10.

Analysis required for C₄₄H₈₄N₂O₆ (737.16): C 71.69%, H 11.49%, N 3.80%; found: C 70.85%, H 11.11%, N 3.81%.

### EXAMPLE 12a

### Preparation of an O-octyl Sterically Hindered Amine Ether (Same Product as in Examples 10-12) Without Isolation of the Intermediate, i.e. Without Solvent Change

t-Butylhydroperoxide (70% in water; 2.1 g, 16.3 mmol) is added at 60° C. within 15 minutes to a stirred mixture of bis(1-oxyl-2,2,6,6-tetramethylpiperidine-4-yl)sebacate (Prostab® 5415, Ciba Specialty Chemicals Inc.; 4.6 g, 9.0 mmol), 1-octene (97%; 20.8 g, 179.8 mmol) and Bu₄NI (0.067 g, 0.18 mmol). The temperature of the reaction mixture is held at 60° C. for a total of 4.5 hours. The yellow emulsion is cooled down to 25° C. and the water-phase split off. The organic phase is dried over Na₂SO₄, filtered and hydrogenated over Pt (10% on carbon; 0.18 g, 0.09 mmol) at 50° C./8 bar. Filtration and evaporation of volatiles leaves the product as yellowish oil exhibiting the same ¹H-NMR spectrum as in example 10.

Analysis calcd. for C₄₄H₈₄N₂O₆ (737.16): C 71.69%, H 11.49%, N 3.80%; found: C 70.78%, H 11.10%, N 3.71%.

### EXAMPLE 13

### Preparation of an O-hexenyl Sterically Hindered Amine Ether from the Corresponding the Corresponding Nitroxide and 1-n-hexene with a tert-BuOOH/CuBr₂ catalyst System

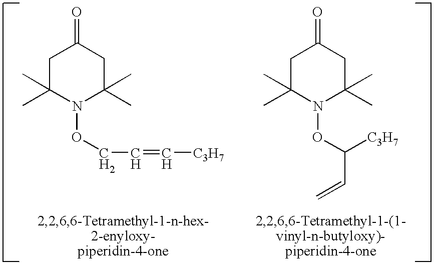

To a stirred mixture of 13.62 g (80 mmol) 2,2,6,6-tetramethyl-4-piperidon-N-oxide, 67.3 g (800 mmol) 1-n-hexene and 0.179 g (0.8 mmol) CuBr₂ are added at reflux (ca 60° C.) within 1.1 hours 15.45 g (120 mmol) tert-butylhydroperoxide (70% aqueous solution). The temperature of the reaction mixture is held at reflux for 2.8 hours. The green emulsion is cooled down to 25° C. and stirred with 75 g of an aqueous 20% Na₂SO₃ solution until the disappearance of excess tert-butylhydroperoxide. The aqueous phase is then separated and washed with pentane. The combined organic phases are washed with water, dried over MgSO₄, filtered and the solvent distilled off on a rotary-evaporator to give 17.75 g (88% of theory) of a yellow liquid. Purification by flash-chromatography (silica gel, hexane/ethylacetate 9/1) affords 12.8 g (63% of theory) of a mixture of 2,2,6,6-tetramethyl-1-n-hex-2-enyloxy-piperidine-4-one (ca 40 mol % by ¹H-NMR) and 2,2,6,6-tetramethyl-1-(1-ethyl-nbutyloxy)-piperidine-4-one (ca 60 mol % by ¹H-NMR).

Analysis required for C₁₅H₂₇NO₂ (253.38): C 71.10%, H 10.74%, N 5.53%; found: C 69.82%, H 10.53%, N 5.25%.

¹H-NMR (CDCl₃), δ (ppm, O—C(n)Hₓ only): 4.16 (q-like, O—C(3)H), 4.31 and 4.42 (d-like, O—C(1)H₂).

¹³C(DEPT)-NMR (CDCl₃), δ (ppm, O—CHₓ and ═CH_{y} only): 73.5 (O—CH₂), 78.5 (O—CH₂), 86.0 (O—CH), 116.5 (═CH₂), 124.6 (═CH), 125.0 (═CH), 133.4 (═CH), 134.5 (═CH), 140.4 (═CH).

### EXAMPLE 14

### Preparation of an O-hexyl Sterically Hindered Amine Ether by Hydrogenation of the O-hexenyl Sterically Hindered Amine Ether (Product of Example 13)

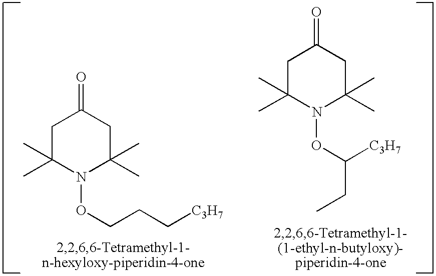

A mixture of 12.8 g (50.5 mmol) product of example 13 and 0.9 g Pt on charcoal (10%) in 120 ml ethylacetate is hydrogenated at 25° C. and 4 bar hydrogen pressure. Filtration and evaporation of the solvent yields 10.48 g (81.2% of theory) of a slightly reddish oil consisting of a mixture of 2,2,6,6-tetramethyl-1-n-hexyloxy-piperidin-4-one (ca 40 mol % by ¹H-NMR) and 1-(1-ethyl-n-butyloxy)-2,2,6,6-tetramethyl-piperidin-4-one (ca 60 mol % by ¹H-NMR).

Analysis required for C₁₅H₂₉NO₂ (255.40): C 70.54%, H 11.44%, N 5.48%; found: C 70.33%, H 11.10%, N 5.37%.

¹H-NMR (CDCl₃), δ (ppm, O—C(n) H, only): 3.76 (p-like, O—C(3)H), 3.82 (t, J=ca 6.6 Hz, O—C(1)H₂).

¹³C(DEPT)-NMR (CDCl₃), δ (ppm, O—C(n)Hₓ only): 77.1 (O—C(1)H₂), 83.4 (O—C(3)H).

### EXAMPLE 15

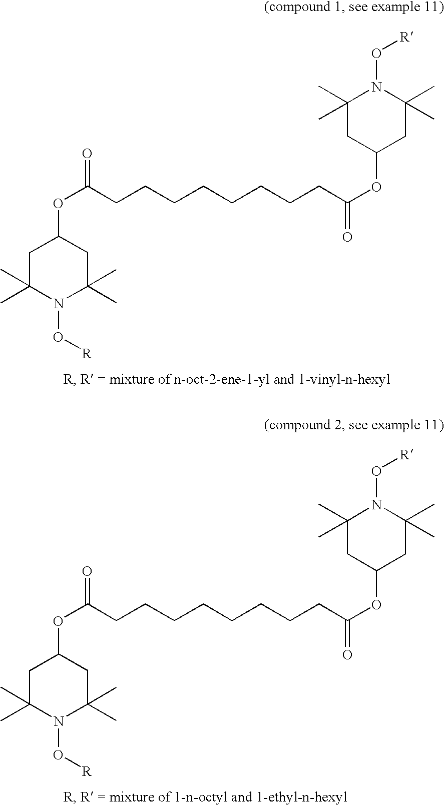

The amino ethers of the present invention are incorporated into a thermosetting acryl/melamine clear coat (based on Viacryl® SC 303/Viacryl® SC 370/Maprenal® MF 650) in a concentration of 1% based on the solids content of the formulation (solids content: 50.4%). The clear coat is sprayed onto silver metallic base coat resulting after cure (130° C./30′) in a dry film thickness of the clear coat of 40 μm. As a substrate electro coated aluminium panels (10×30 cm) as commercially available from ACT Laboratories (ACT Laboratories, Inc., Southfield, Mich. 48 075, USA) are being used. The panels are subsequently exposed in a Xenon—WOM wetherometer (Atlas Corp.) according to SAE J 1960. The 20° gloss is recorded in regular intervals. The test results are summarized in Table 1:

TABLE 1

| | 20° Gloss | 20° Gloss after | 20° Gloss after |
|---|---|---|---|
| Sample | Initial | 2000 h exposure | 4000 h exposure |
| No additive | 90 | 60 | 21 (end with |
| | | | cracking) |
| 1% Compound 1 | 90 | 77 | 56 |
| 1% Compound 2 | 90 | 73 | 62 |

### EXAMPLE 16

The experiments are performed as described in example 15, however, the amino ethers of the present invention are now tested in combination with 1.5% UV—absorber 1 (TINUVIN® 384) (concentration based on the solids content of the clear coat formulation). The results are summarized in Table 2:

TABLE 2

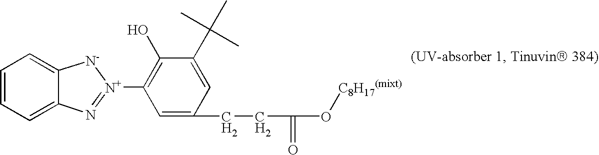

| | 20° Gloss | 20° Gloss after | 20° Gloss after | 20° Gloss after |
|---|---|---|---|---|
| Sample | Initial | 2000 h exposure | 4000 h exposure | 6000 h exposure |
| No additive | 90 | 60 | 21 (end with | |
| | | | cracking) | |
| 1% Compound 1 + | 90 | 80 | 76 | 70 |
| 1.5% UV-absorber 1 | | | | |
| 1% Compound 2 + | 90 | 78 | 74 | 60 |
| 1.5% UV-absorber 1 | | | | |
| 1.5% UV-absorber 1 | 90 | 77 | 59 (end with | |
| alone | | | cracking) | |

Clearcoat formulation:

| | | |
|---|---|---|
| a) | Viacryl SC 303: acrylic resin (Solutia, formerly | 27.51 g |
| | Vianova Resins) (65% solution in xylene/butanol, | |
| | 26:9 wt./wt) | |
| b) | Viacryl SC 370: acrylic resin (Solutia, formerly | 23.34 g |
| | Vianova Resins) (75% in Solvesso 100: aromatic | |
| | hydrocarbon, bp. 163-180° C. (Exxon Corp.)) | |
| c) | Maprenal MF 650: melamine resin (Solutia, formerly | 27.29 g |
| | Vianova Resins) (55% in isobutanol) | |
| d) | Butylacaetate/butanol (37:8 wt./wt.) | 4.33 g |
| e) | Isobutanol | 4.87 g |
| f) | Solvesso 150: aromatic hydrocarbon, bp. 180-203° C. | 2.72 g |
| | (Exxon Corp.) | |
| g) | Crystal oil 30: aliphatic hydrocarbon, bp. 145-200° C. | 8.74 g |
| | (Shell Corp.) | |
| h) | Baysilone MA: leveling agent (Bayer AG) (1% in | 1.20 g |
| | Solvesso 150) | |
| | Total | 100.00 g |

1. A process for the preparation of a sterically hindered amine ether which comprises reacting a corresponding sterically hindered aminoxide with a C₅-C₁₈alk-1-ene in the presence of an organic hydroperoxide.

2. A process according to claim 1, wherein the obtained product is subsequently hydrogenated.

3. A process according to claim 1, wherein the sterically hindered amine oxide contains at least one group of formula (II) 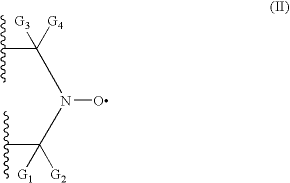
wherein G₁, G₂, G₃ and G₄ are independently alkyl of 1 to 4 carbon atoms or G₁ and G₂ and/or G₃ and G₄ are together tetramethylene or pentamethylene.

4. A process according to claim 1, wherein the obtained sterically hindered amine ether contains at least one group of formula (III) 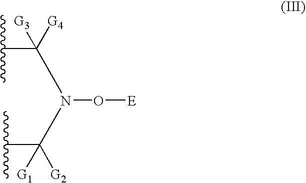
wherein G₁, G₂, G₃ and G₄ are independently alkyl of 1 to 4 carbon atoms or G₁ and G₂ and/or G₃ and G₄ are together tetramethylene or pentamethylene and
C₅-C₁₈alkyl or C₅-C₁₈alk-2-enyl.

5. A process according to claim 3, wherein G₁ and G₃ are methyl and G₂ and G₄ are independently methyl or ethyl.

6. A process according to claim 4, wherein the sterically hindered amine ether is of formula (A) to (O) 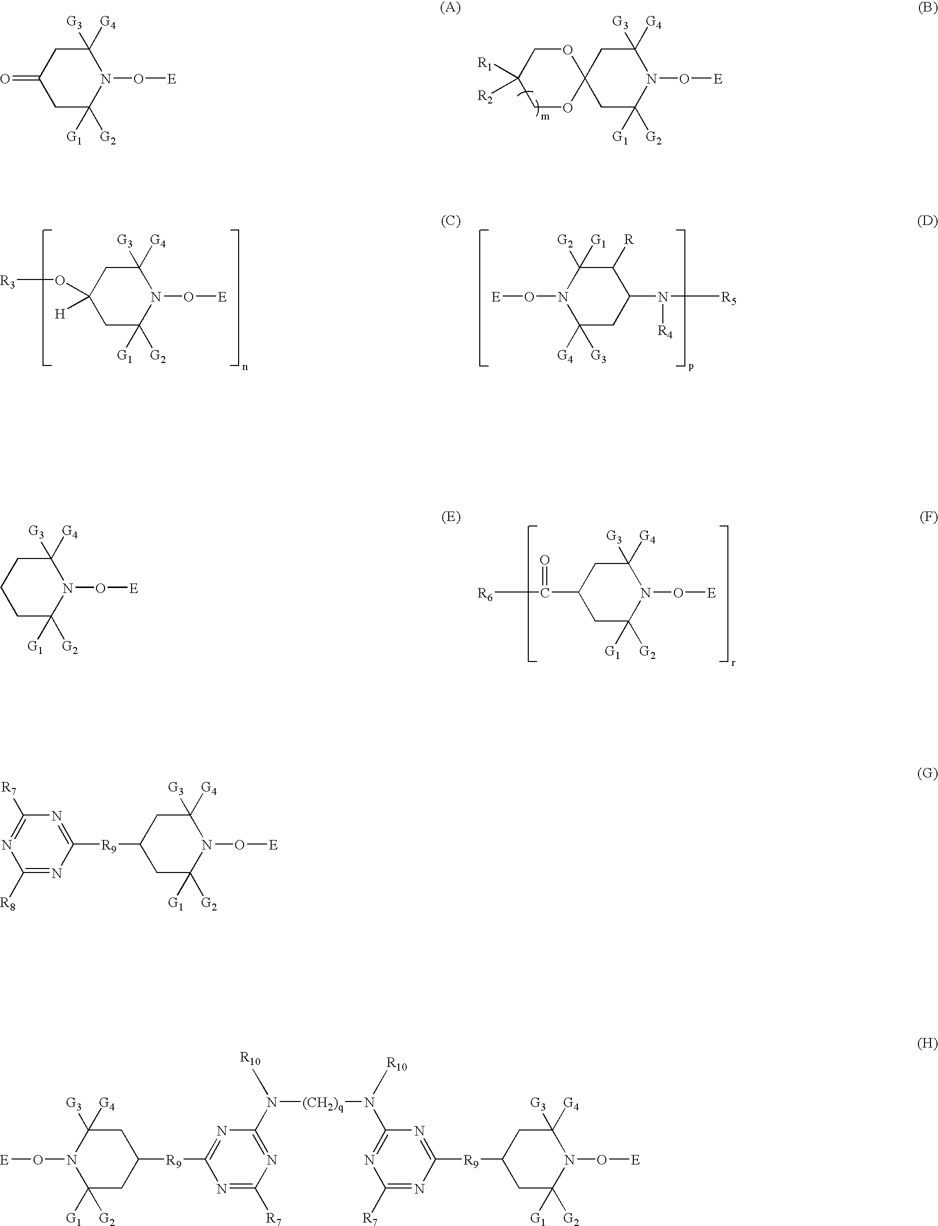 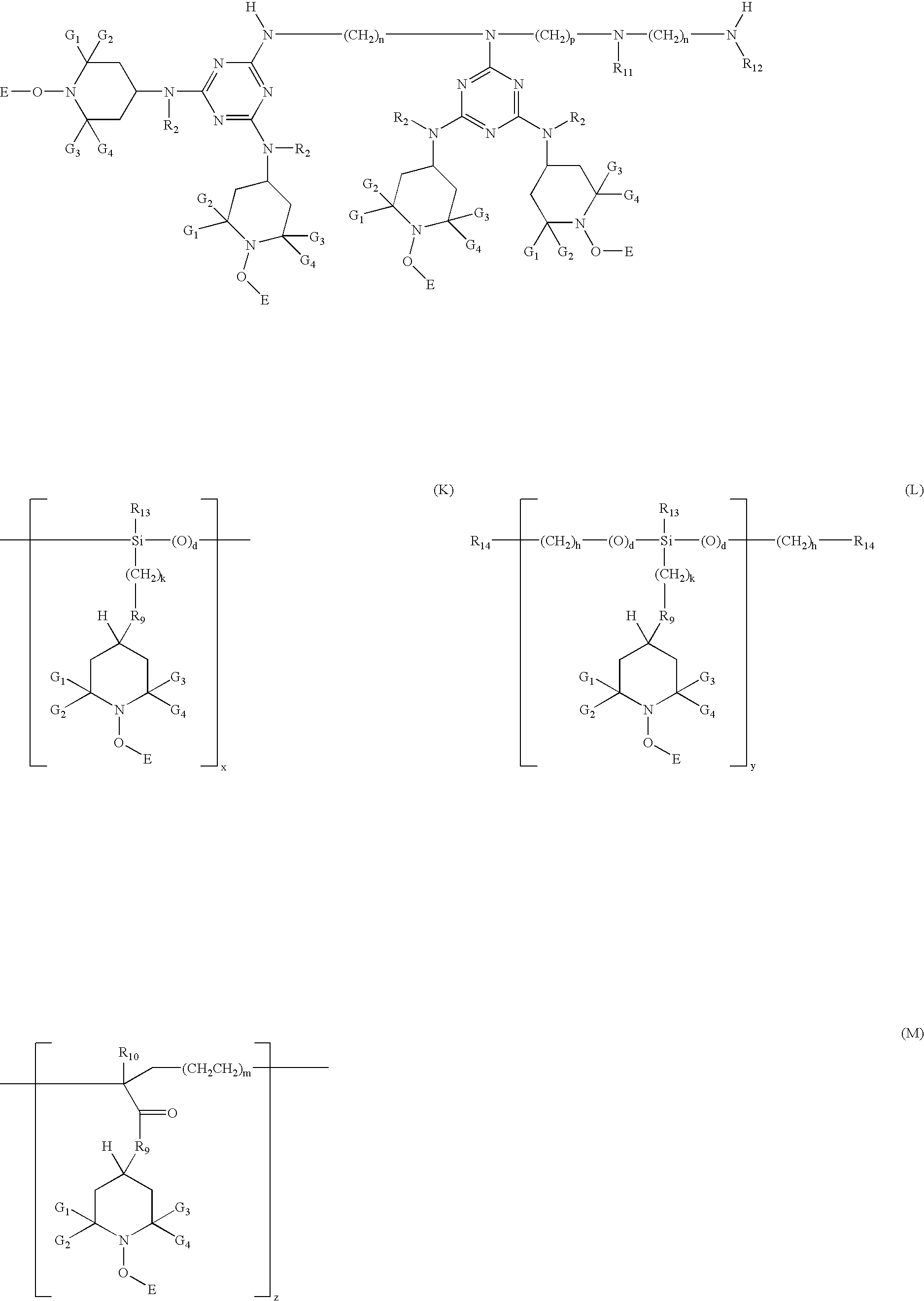 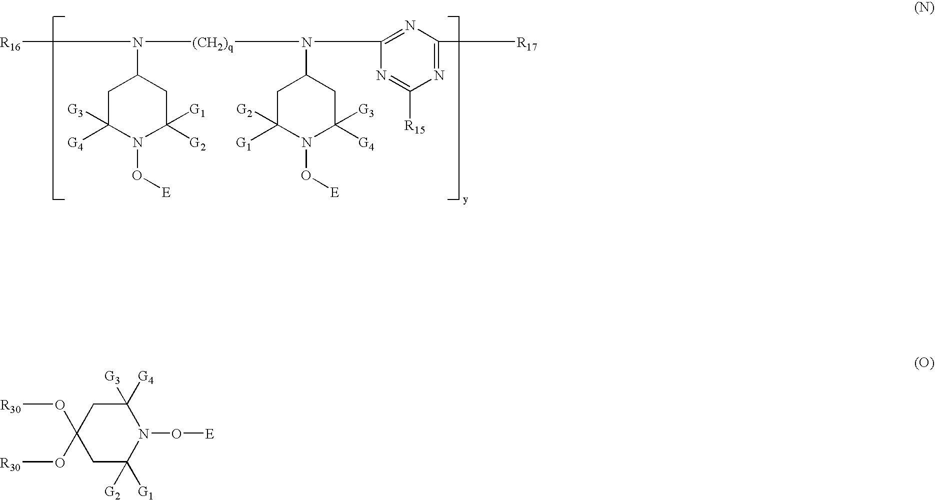
wherein G₁, G₂, G₃ and G₄ are independently alkyl of 1 to 4 carbon atoms or G₁ and G₂ and/or G₃ and G₄ are together tetramethylene or pentamethylene and
E is C₅-C₁₈alkyl or C₅-C₁₈alk-2-enyl;
m is 0 or 1;
R₁ is hydrogen, hydroxyl or hydroxymethyl;
R₂ is hydrogen, alkyl of 1 to 12 carbon atoms or alkenyl of 2 to 12 carbon atoms;
n is 1 to 4;
when n is 1,
R₃ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₃ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₃ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₃ is a tetravalent acyl radical of an aliphatic or unsaturated aliphatic tetracarboxylic acid or R₃ is a tetravalent acyl radical of an aromatic tetracarboxylic acid containing 10 to 18 carbon atoms;
p is 1 to 3,
R₄ is hydrogen, alkyl of 1 to 18 carbon atoms or acyl of 2 to 6 carbon atoms or phenyl;
when p is 1,
R₅ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;
when p is 2,
R₅ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4,
when r is 1,
R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,
when r is 2,
R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₆ is 4-methyl-1,3-phenylenediamino,
when r is 3,
R₆ is a trivalent alkoxy radical of a saturated or unsaturated aliphatic triol containing 3 to 18 carbon atoms,
when r is 4,
R₆ is a tetravalent alkoxy radical of a saturated or unsaturated aliphatic tetraol containing 4 to 18 carbon atoms,
R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms, R₉ is oxygen, or R₉ is nitrogen substituted by either hydrogen, alkyl of 1 to 12 carbon atoms or T₁,
T₁ is 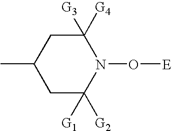
R₁₀ is hydrogen or methyl,
q is 2 to 8,
R₁₁ and R₁₂ are independently hydrogen or the group T₂,
T₂ is 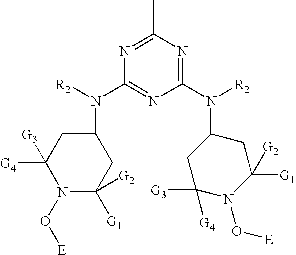
R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₄ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₅ is morpholino, piperidino, 1-piperizinyl, alkylamino of 1 to 8 carbon atoms, —N(alkyl)T₁ with alkyl of 1 to 8 carbon atoms, or —N(alkyl)₂ of 2 to 16 carbon atoms,
R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents may be different;
R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃,
T₃ is 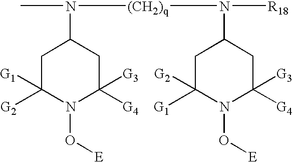
R₁₈ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms;
R₃₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms.

7. A process according to claim 1, wherein the C₅-C₁₈alk-1-ene is C₆-C₁₂alk-1-ene.

8. A process according to claim 1, wherein the reaction is carried out in the presence of a further catalyst.

9. A process according to claim 8, wherein the further catalyst is selected from the group consisting of scandium, titanium, vanadium, chromium, manganese, iron, cobalt, nickel, copper, zinc, gallium, germanium, yttrium, zirconium, niobium, molybdenum, ruthenium, rhodium, palladium, silver, cadmium, indium, tin, antimony, lanthanum, cerium, hafnium, tantalum, tungsten, rhenium, osmium, iridium, platinum, gold, mercury, thallium, lead, bismuth; the compounds thereof; ammonium iodides and phosphonium iodides.

10. A process according to claim 8, wherein the further catalyst is selected from the group consisting of titanium, vanadium, chromium, manganese, iron, cobalt, nickel, copper, zinc, cerium; the halides and oxides thereof; ammonium iodides and phosphonium iodides.

11. A process according to claim 1, wherein the organic hydroperoxide contains 3-18 carbon atoms.

12. A process according to claim 2, wherein the hydrogenation is carried out in the presence of a hydrogenation catalyst.

13. A process according to claim 12, wherein the hydrogenation catalyst is selected from the group consisting of platinum, palladium, ruthenium, rhodium, Lindlar catalyst, platinum compounds, palladium compounds, ruthenium compounds, rhodium compounds, iridium compounds, nickel compounds, zinc compounds and cobalt compounds.

14. A mixture of sterically hindered amine ethers of formula (Za) 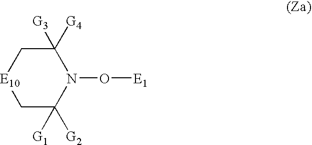
wherein G₁, G₂, G₃ and G₄ are independently alkyl of 1 to 4 carbon atoms or G₁ and G₂ and/or G₃ and G₄ are together tetramethylene or pentamethylene; E₁₀ is a carbon atom which is unsubstituted or substituted by —OH, ═O or by one or two organic residues containing in total 1-500 carbon atoms and
E₁ is a mixture of the radicals 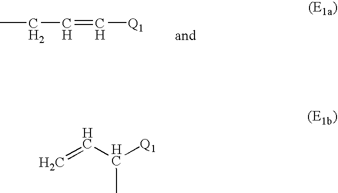
wherein Q₁ is C₂-C₁₅ alkyl;
with the proviso that the mixture of sterically hindered amine ethers is not a mixture of compounds of formula (100) and (101)

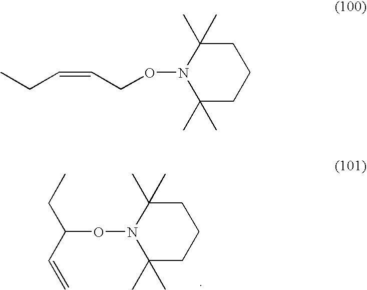

15. A mixture according to claim 14 of sterically hindered amine ethers represented by formulae (A) to (O) 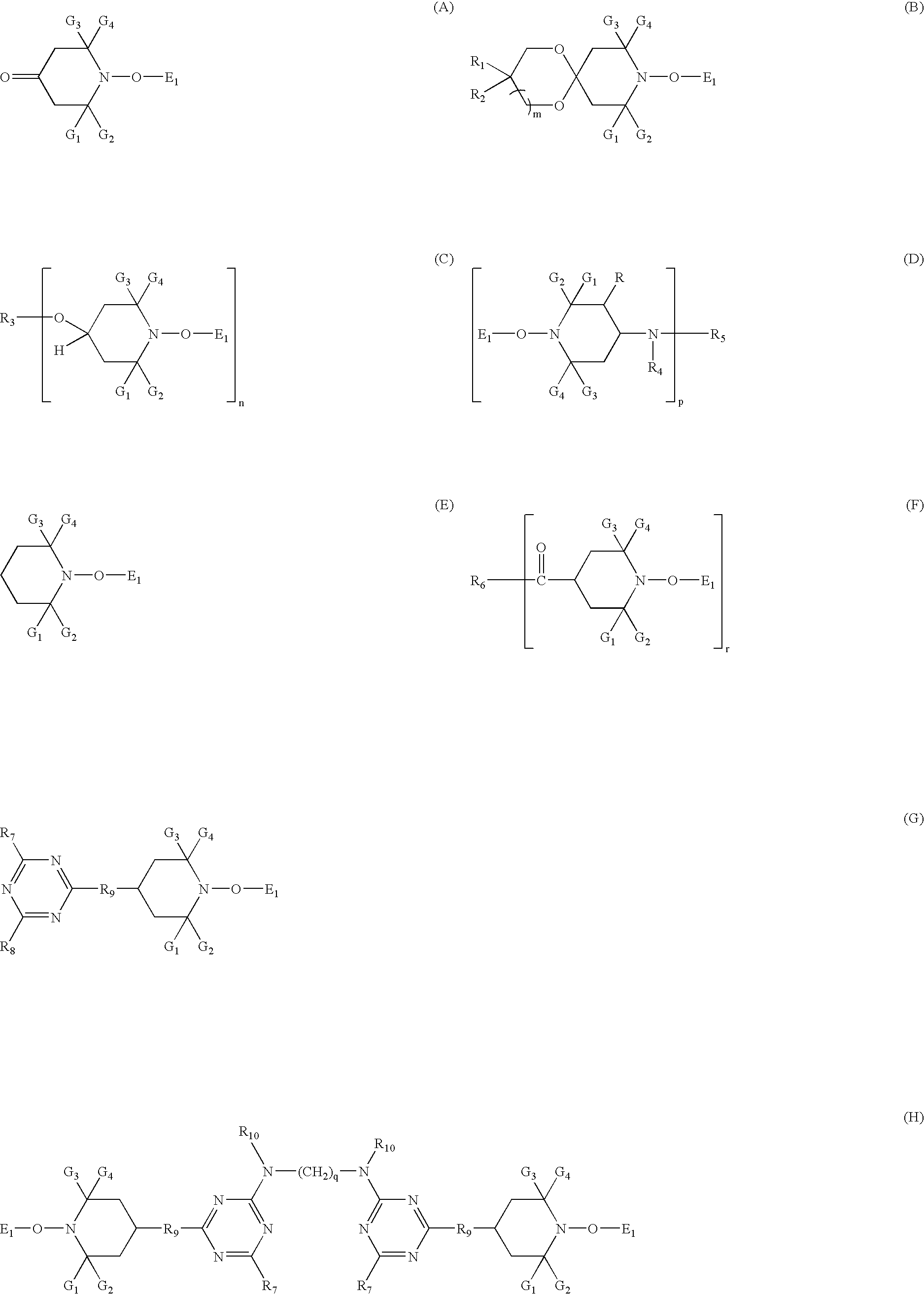 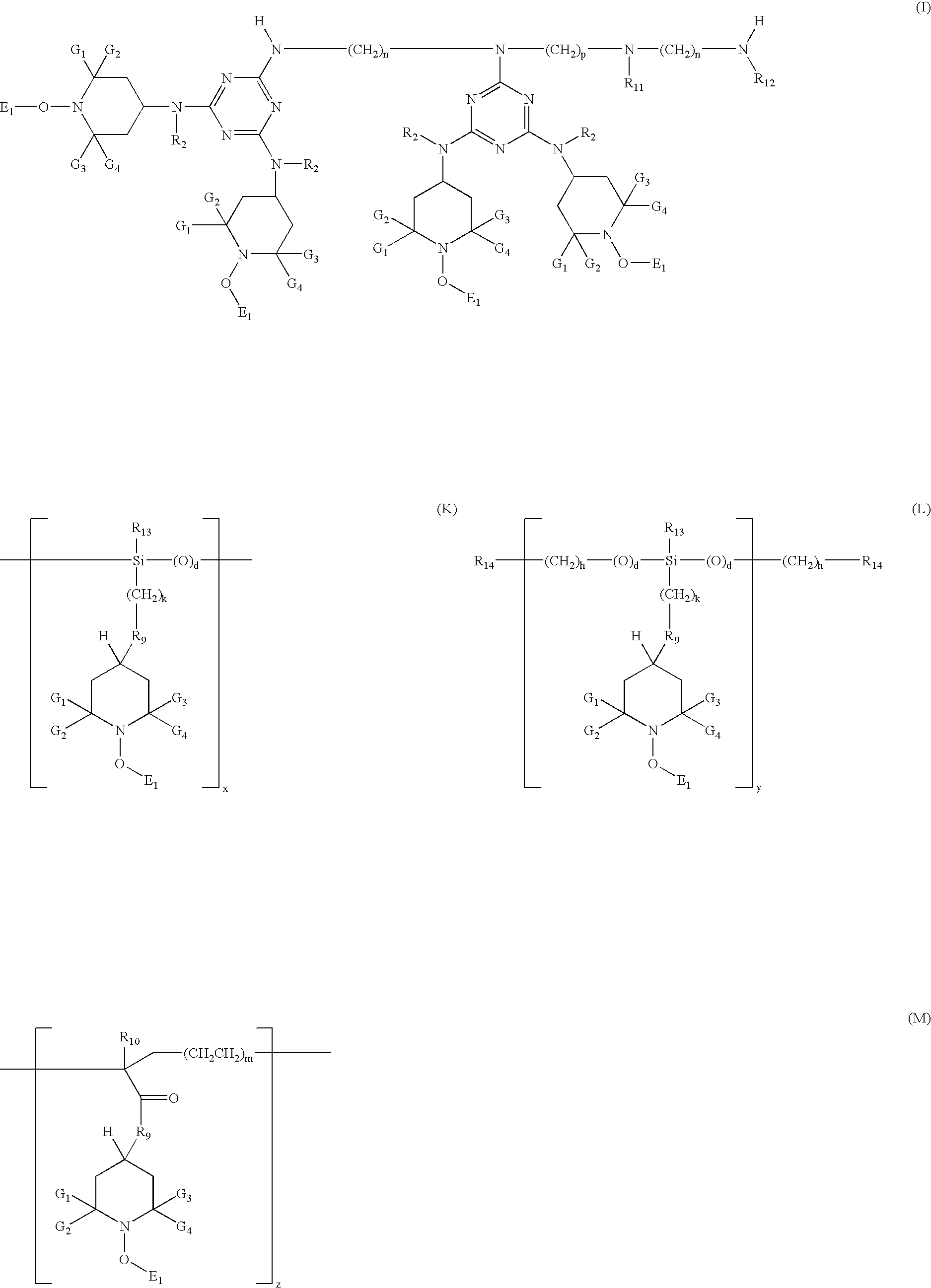 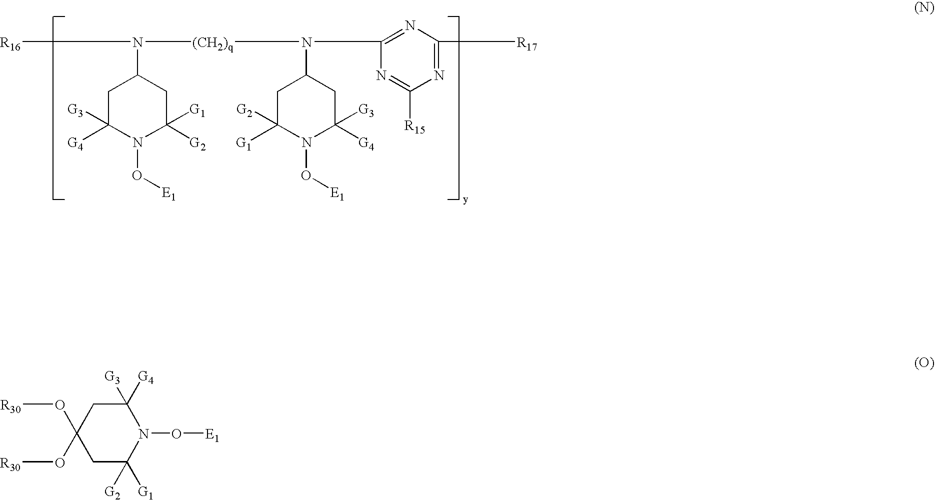
wherein G₁, G₂, G₃ and G₄ are independently alkyl of 1 to 4 carbon atoms or G₁ and G₂ and/or G₃ and G₄ are together tetramethylene or pentamethylene;
m is 0 or 1;
R₁ is hydrogen, hydroxyl or hydroxymethyl:
R₂ is hydrogen, alkyl of 1 to 12 carbon atoms or alkenyl of 2 to 12 carbon atoms:
n is 1 to 4,
when n is 1,
R₃ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₃ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₃ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4.
R₃ is a tetravalent acyl radical of an aliphatic or unsaturated aliphatic tetracarboxylic acid or
R₃ is a tetravalent acyl radical of an aromatic tetracarboxylic acid containing 10 to 18 carbon atoms;
p is 1 to 3,
R₄ is hydrogen, alkyl of 1 to 18 carbon atoms or acyl of 2 to 6 carbon atoms or phenyl;
when p is 1,
R₅ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;
when p is 2,
R₅ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4,
when r is 1,
R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,
when r is 2,
R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₆ is 4-methyl-1,3-phenylenediamino,
when r is 3,
R₆ is a trivalent alkoxy radical of a saturated or unsaturated aliphatic triol containing 3 to 18 carbon atoms,
when r is 4,
R₆ is a tetravalent alkoxy radical of a saturated or unsaturated aliphatic tetraol containing 4 to 18 carbon atoms,
R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms,
R₉ is oxygen, or R₉ is nitrogen substituted by either hydrogen, alkyl of 1 to 12 carbon atoms or T₁,
T₁ is 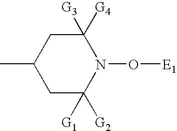
R₁₀ is hydrogen or methyl,
q is 2 to 8,
R₁₁ and R₁₂ are independently hydrogen or the group T₂,
T₂ is 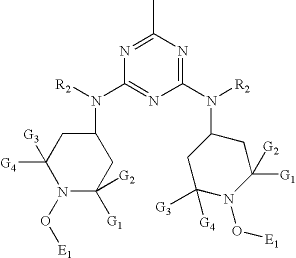
R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₄ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
v is 1 to 10;
z is an integer such that the compound has a molecular weight of 1000 to 4000 amu, e.g. z may be from the range 3-10;
R₁₅ is morpholino, piperidino, 1-piperizinyl, alkylamino of 1 to 8 carbon atoms, —N(alkyl)T₁ with alkyl of 1 to 8 carbon atoms, or —N(alkyl)₂ of 2 to 16 carbon atoms,
R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents may be different;
R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃,
T₃ is 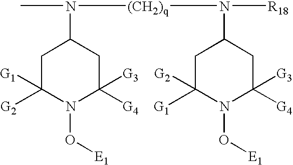
R₁₈ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms;
R₃₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms.

16. A mixture of sterically hindered amine ethers containing at least one group of formula (Y) 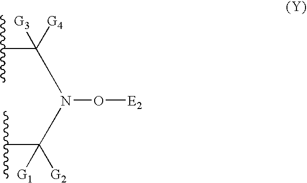
wherein G₁, G₂, G₃ and G₄ are independently alkyl of 1 to 4 carbon atoms or G₁ and G₂ and/or G₃ and G₄ are together tetramethylene or pentamethylene and
E₂ is a mixture of the radicals 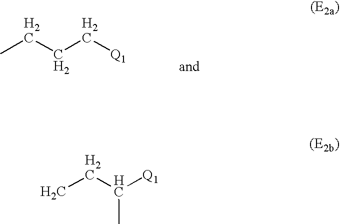
wherein Q₁ is C₂-C₁₅ alkyl.

17. A mixture according to claim 16 of sterically hindered amine ethers represented by formulae (A) to (O) 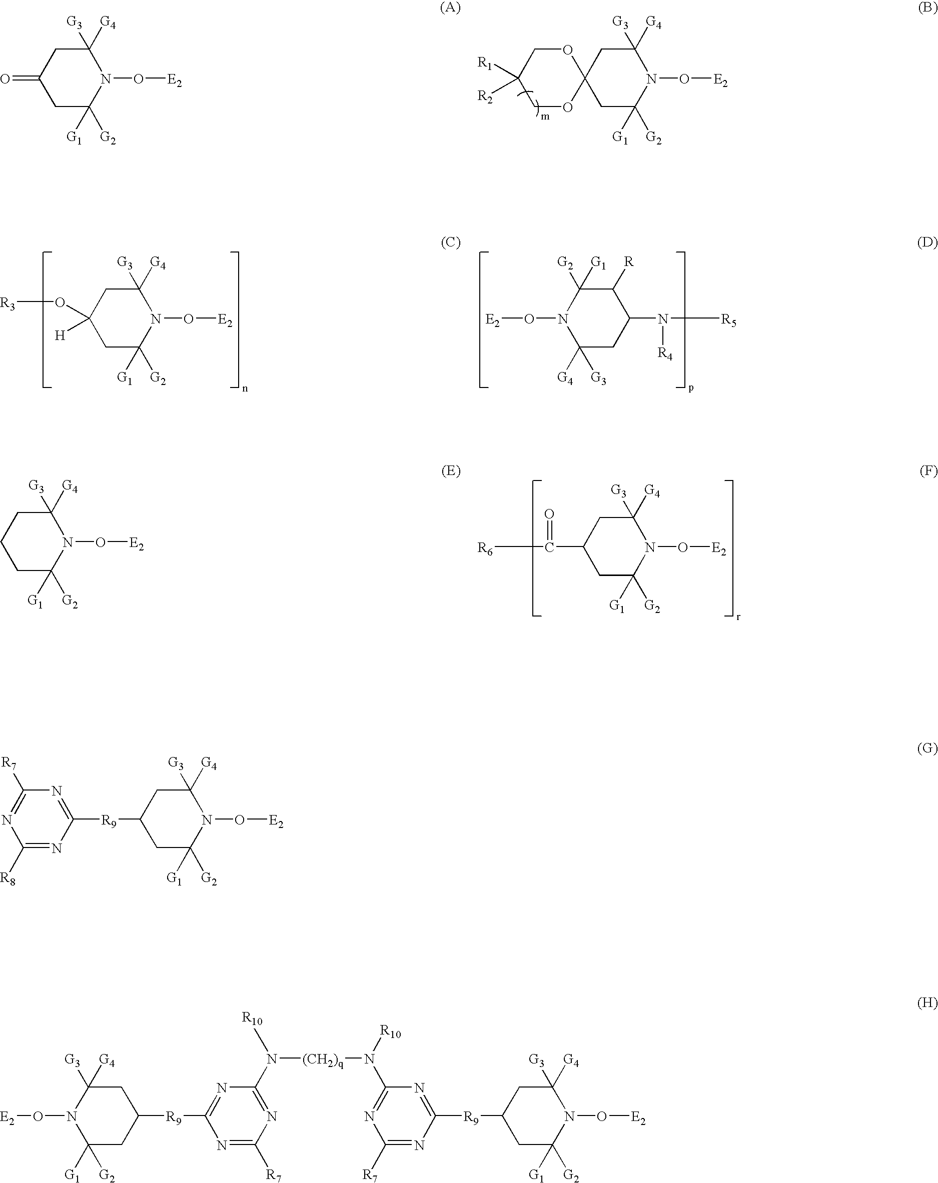 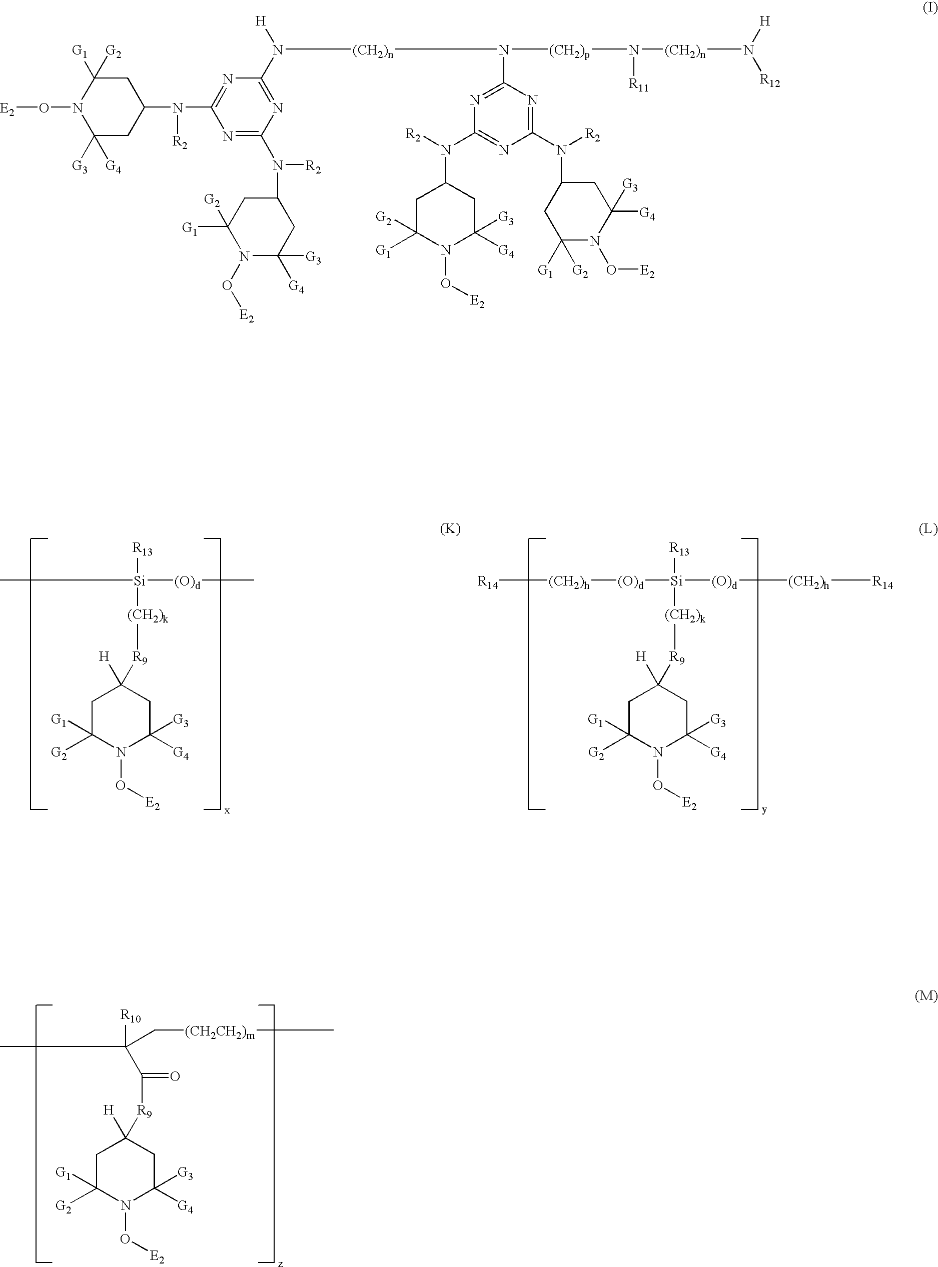 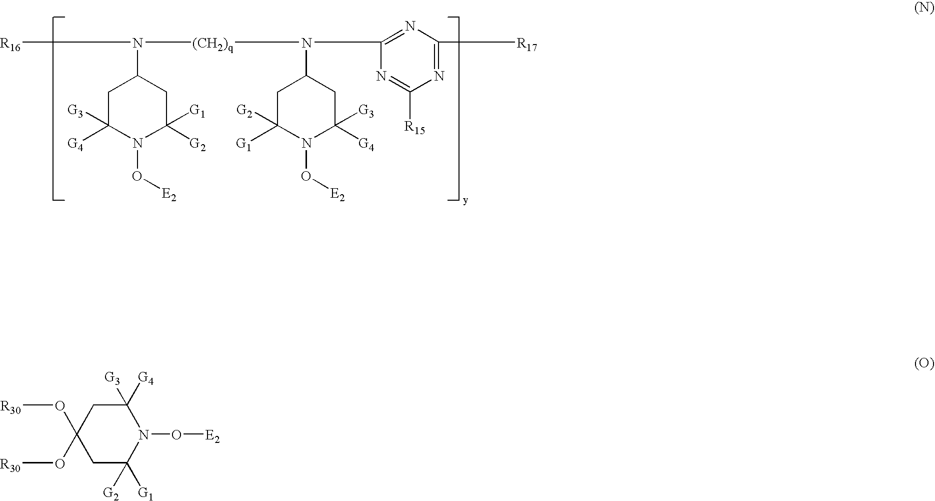
wherein G₁, G₂, G₃ and G₄ are independently alkyl of 1 to 4 carbon atoms or G₁ and G₂ and/or G₃ and G₄ are together tetramethylene or pentamethylene;
m is 0 or 1;
R₁ is hydrogen, hydroxyl or hydroxymethyl:
R₂ is hydrogen, alkyl of 1 to 12 carbon atoms or alkenyl of 2 to 12 carbon atoms;
n is 1 to 4;
when n is 1,
R₃ is hydrogen, alkyl of 1 to 18 carbon atoms, alkoxycarbonylalkylenecarbonyl of 4 to 18 carbon atoms, alkenyl of 2 to 18 carbon atoms, glycidyl, 2.3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₃ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₃ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₃ is a tetravalent acyl radical of an aliphatic or unsaturated aliphatic tetracarboxylic acid or
R₃ is a tetravalent acyl radical of an aromatic tetracarboxylic acid containing 10 to 18 carbon atoms;
p is 1 to 3,
R₄ is hydrogen, alkyl of 1 to 18 carbon atoms or acyl of 2 to 6 carbon atoms or phenyl;
when p is 1,
R₅ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;
when p is 2.
R₅ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4,
when r is 1,
R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,
when r is 2,
R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₆ is 4-methyl-1,3-phenylenediamino,
when r is 3,
R₆ is a trivalent alkoxy radical of a saturated or unsaturated aliphatic triol containing 3 to 18 carbon atoms,
when r is 4,
R₆ is a tetravalent alkoxy radical of a saturated or unsaturated aliphatic tetraol containing 4 to 18 carbon atoms,
R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms,
R₉ is oxygen, or R₉ is nitrogen substituted by either hydrogen, alkyl of 1 to 12 carbon atoms or T₁,
T₁ is 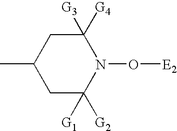
R₁₀ is hydrogen or methyl.
q is 2 to 8,
R₁₁ and R₁₂ are independently hydrogen or the group T₂,
T₂ is 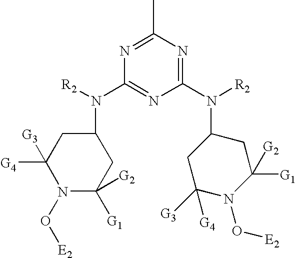
R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₄ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
v is 1 to 10;
z is an integer such that the compound has a molecular weight of 1000 to 4000 amu, e.g. z may be from the range 3-10;
R₁₅ is morpholino, piperidino, 1-piperizinyl, alkylamino of 1 to 8 carbon atoms, —N(alkyl)T₁ with alkyl of 1 to 8 carbon atoms, or —N(alkyl)₂ of 2 to 16 carbon atoms,
R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents may be different;
R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃,
T₃ is 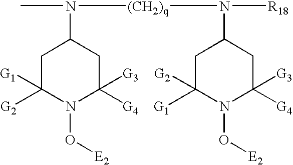
R₁₈ is hydrogen, acyl of 2 to 4 carbon atoms, carbamoyl substituted by alkyl of 1 to 4 carbon atoms, s-triazinyl substituted twice by —N(alkyl), of 2 to 16 carbon atoms or s-triazinyl substituted twice by —N(alkyl)T₁ with alkyl of 1 to 8 carbon atoms;
R₃₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms.

18. A mixture according to claim 14, wherein the ratio E₁ₐ:E_{1b} is from 1:9 to 7:3.

19. A process for flame retarding an organic material or stabilizing an organic material against degradation by light, oxygen and/or heat, which process comprises applying to or incorporating into said material a mixture of sterically hindered amine ethers as defined in claim 14.

20. A composition comprising
A) an organic material which is sensitive to oxidative, thermal and/or actinic degradation, and
B) at least one mixture of sterically hindered amine ethers as defined in claim 14.

21. A composition according to claim 20, comprising further additives.

22. A composition according to claim 21, comprising as further additives antioxidants, UV-absorbers, light stabilizers, metal deactivators, phosphites, phosphonites, hydroxylamines, nitrones, thiosynergists, peroxide scavengers, basic co-stabilizers, nucleating agents, fillers, reinforcing agents, benzofuranones, indolinones and/or flameproofing agents.
